(19) United States
(12) Patent Application Publication
(10) Pub. No.: US 2005/0220819 A1
(43) Pub. Date: Oct. 6, 2005

(54) Novel binding epitopes for helicobacter pylori and use thereof

(75) Inventors: Halina Miller-Podraza, Frolunda (SE); Susann Teneberg, Hindas (SE); Jonas Angstrom, Goteborg (SE); Karl-Anders Karlsson, Goteborg (SE); Jari Natunen, Vantaa (FI); Jari Helin, Vantaa (FI)

Correspondence Address: BIRCH STEWART KOLASCH & BIRCH, PO BOX 747, FALLS CHURCH, VA, 22040-0747, US

(73) Assignee: Biotie Therapies OYJ

(21) Appl. No.: 10/501,786

(22) PCT Filed: Jan. 20, 2003
(86) PCT No.: PCT/FI03/00039

§ 371 Date: May 17, 2005

(30) Foreign Application Priority Data
Jan. 18, 2002 (WO) PCT/FI02/00043

(51) Int. Cl. C07K 14/195; A61K 39/02; C08B 37/00

(52) U.S. Cl. 424/234.1; 530/395; 536/53

(57) ABSTRACT

The present application describes a binding epitope to Helicobacter pylori, namely substances comprising the oligosaccharide sequence -Gal(Nac)ᵣ₂$g(b)4Glc(A)_{q2}(Nac)ᵣ₃-, and the use thereof in pharmaceutical and nutritional compositions for the treatment of conditions due to the presence of Helicobacter pylori in a subject, e.g. gastritis, gastric ulcers, gastric adenocarcinoma, liver disease, pancreatic disease, skin disease, heart disease, autoimmune diseases and sudden infant death syndrome. The invention is also directed to the use of the receptor for diagnostics of Helicobacter pylori, to a method of producing chondroitin oligosaccharides from chondroitin sulphates, and a method for production of amidated glucuronic acid comprising oligosaccharides and monosaccharides from glucuronic acid comprising polysaccharides.

### FIELD OF THE INVENTION

The present invention describes a substance or receptor binding to Helicobacter pylori, and use thereof id e.g., pharmaceutical and nutritional compositions for the treatment of conditions due to the presence of Helicobacter pylori. The invention is also directed to the use of the receptor for diagnostics of Helicobacter pylori.

### BACKGROUND OF THE INVENTION

Helicobacter pylori has been implicated in several diseases of the gastrointestinal tract including chronic gastritis, non-steroidal anti-inflammatory drug (NSAID) associated gastric disease, duodenal and gastric ulcers, gastric MALT lymphoma, and gastric adenocarcinoma (Axon, 1993; Blaser, 1992; DeCross and Marshall, 1993; Dooley, 1993; Dunn et al., 1997; Lin et al., 1993; Nomura and Stemmermann, 1993; Parsonnet et al. 1994; Sung et al., 2000 Wotherspoon et al., 1993). Helicobacter pylori is also involved in totally or partially non-gastrointestinal diseases including sudden infant death syndrome (Kerr et al., 2000 and U.S. Pat. No. 6,083,756), autommune diseases such as autoimmune gastritis and pernicious anaemia (Appelmelk et al., 1998; Chmiela et al, 1998; Clayes et al., 1998; Jassel et al., 1999; Steininger et al., 1998) and some skin diseases (Rebora et al., 1995), pancreatic disease (Correa et al., 1990), liver diseases including adenocarcinoma (Nilsson et al., 2000; Avenaud et al., 2000) and heart diseases such as atherosclerosis (Farsak et al., 2000). Multiple diseases caused or associated with Helicobacter pylori has been reviewed (Pakodi et al., 2000). Of prime interest with respect to bacterial colonization and infection is the mechanism by which this bacterium adheres to the epithelial cell surfaces of the gastric mucosa.

Glycoconjugates, both lipid- and protein-based, have been reported to serve as receptors for the binding of this microorganism, e.g., sialylated glycoconjugates (Evans et al., 1988), sulfatide and GM3, NeuNAcα3LacβCer, (Saitoh et al., 1991), Le^{b} determinants, Fucα2Galβ3(Fucα4)GlcNAc (Borén et al., 1993), polyglycosylceramides, heterogenous branched and linear fucosylated and sialylated structures with sialic acid as part of receptor (Miller-Podraza et al., 1996; 1997a), lactosylceramide, LacβCer(hydroxyl fattyacids) (Ångström et al., 1998) and gangliotetraosylceramide (Lingwood et al., 1992; Ångström et al., 1998). Other potential receptors for Helicobacter pylori include polysaccharide heparan sulphate, (GlcNAcα4GlcAβ/IdoAα4)ₙ comprising various sulphates, (Ascensio et al., 1993) as well as the phospholipid phosphatidylethanolamine (Lingwood et al., 1992). These sequences are different from the epitopes described by the present invention. The polysaccharides chondroitin sulphate, chondroitin, and hyaluronic acid, and even non-specified fragments derived from these as polyvalent conjugates, have been also described as receptors for Helicobacter pylori (FI20011403). However, FI20011403 does not describe minimum size of the oligosaccharide effective for inhibition or binding of H. pylori (if any possible in monovalent form) and does not give effective binding structure. Moreover, the exact sulphation status of the chondroitin oligosaccharides was not described nor possible natural variations of the oligosaccharide sequences or the role of these in specific binding to H. pylori.

Numerous patent documents describe therapheutic compositions comprising chondroitin sulphate, chondroitin sulphate or hyaluronic acid polysaccharides or fragments thereof (e.g. WO09827988, EP0704216, U.S. Pat. No. 5,894,070, U.S. Pat. No. 4,524,666, WO09407505) in various therapheutic compositions. WO9106303 claims chondroitin sulphate as molecules comprising chondroitin sulphate disaccharide, but specific oligosaccharides or non-sulphated oligosaccharides were not claimed. Chondroitin sulphate oligosaccharides has not been described as medicines and specific molecular structures has not been indicated. Some applications describe hyaluronic acid oligosaccharides in medical applications (WO0204471, WO9957301), the latter application describes ester derivatives of hyaluronic acid.

Gangliotetraosysceramide has sequence Galβ3GalNAcβ4Galβ4GlcβCer. An international application WO 02056893 claims similar terminal sequences Galβ3GalNAc of gangliotetraosylceramide and disaccharide terminal Galβ3GlcNAc of lactotetraosylceramide Galβ3GlcNAcβ3Galβ4GlcβCer as H. pylori binding sequences. However, the minimal disaccharide of the present invention GalNAcβ4Glc(A)_{0/1}(NAc)_{0/1} has different monosaccharides and different linkage configuration compared to Galβ3GalNAc.

U.S. patents of Zopf et al.: U.S. Pat. No. 5,883,079 (March 1999), U.S. Pat. No. 5,753,630 (May 1998) and U.S. Pat. No. 5,514,660 (May, 1996) describe Neu5Acα3Gal- containing compounds as inhibitors of the H. pylori adhesion. The sialyl-lactose molecule inhibits Helicobacter pylori binding to human gastrointestinal cell lines (Simon et al., 1999) and is also effective in a rhesus monkey animal model of the infection (Mysore et al., 2000). The compound is in clinical trials.

U.S. patent Krivan et al. U.S. Pat. No. 5,446,681 (November 1995) describes bacterium receptor antibiotic conjugates comprising an asialo ganglioside coupled to a penicillin antibiotic. Especially is claimed the treatment of Helicobacter pylori with an amoxicillin-asialo-GM1 conjugate. The oligosaccharide sequences/glycolipids described by the invention do not belong to the ganglioseries of glycolipids.

U.S. patents of Krivan et al.: U.S. Pat. No. 5,386,027 (January 1995) and U.S. Pat. No. 5,217,715 (June 1993) describe use of oligosaccharide sequences or glycolipids to inhibit several pathogenic bacteria, however the current binding specificity is not included and Helicobacter pylori is not among the bacteria studied or claimed.

The saccharide sequence GlcNAcβ3Gal has been described as a receptor for Streptococcus (Andersson et al., 1986). Some bacteria may have overlapping binding specificities, but it is not possible to predict the bindings of even closely related bacterial adhesins. In case of Helicobacter pylori the saccharide binding molecules, except the Lewis b binding protein are not known.

### SUMMARY OF THE INVENTION

The present invention relates to a substance or receptor binding to Helicobacter pylori comprising the oligosaccharide sequence
[Hex1(A)_{q1}(NAc)ᵣ₁y3]ₛGal(NAc)ᵣ₂β4Glc(A)_{q2}(NAc)ᵣ₃
wherein q1, q2, r1, r2, r3, r5 and s, and w are each independently 0 or 1, and Hex1, and Hex2 is a hexose structures, preferably galactose (Gal) or glucose (Glc) or mannose (Man), most preferably Gal or Glc, which may be further modified by the A and/or NAC groups; y is either alpha or beta indicating the anomeric structure of the terminal monosaccharide residue with the provisions that at least q1, r2 or q2 is 1 and analogs or derivatives of said oligosaccharide sequence for binding or inhibiting Helicobacter pylori.

Among the objects of the invention are the use of the Helicobacter pylori binding oligosaccharide sequences described in the invention as a medicament, and the use of the same for the manufacture of a pharmaceutical composition, particularly for the treatment of any condition due to the presence of Helicobacter pylori in a subject.

The present invention also relates to the methods for the treatment of conditions due to the presence of Helicobacter pylori. The invention is also directed to the use of the receptor(s) described in the invention as Helicobacter pylori binding or inhibiting substance for diagnostics of Helicobacter pylori.

Another object of the invention is to provide substances, pharmaceutical compositions and nutritional additives or compositions containing Helicobacter pylori binding oligosaccharide sequence(s).

Other objects of the invention are the use of the above-mentioned Helicobacter pylori binding substances for the identification of bacterial adhesin the typing of Helicobacter pylori, and the Helicobacter pylori binding assays.

Yet another object of the invention is the use of the above-mentioned Helicobacter pylori binding substances for the production of a vaccine against Helicobacter pylori.

### BRIEF DESCRIPTION OF THE DRAWINGS

FIGS. 1A and 1B. Thin-layer chromatogram with separated glycosphingolipids detected with 4-methoxybenzaldehyde (1A) and autoradiogram after binding of radiolabeled Helicobacter pylori strain 032 (1B). The glycosphingolipids were separated on aluminum-backed silica gel 60 HPTLC plates (Merck) using chloroform/methanol/water 60:35:8 (by volume) as solvent system. The binding assay was done as described in the “Materials and methods” section. Autoradiography was for 72 h. The lanes contained:
lane 1) Galβ4GlcNAcβ3Galβ4Glcβ1Cer (neolactotetraosylceramide), 4 μg;
lane 2) Galα3Galβ4GlcNAcβ3Galβ4Glcβ1Cer (B5 glycosphingolipid), 4 μg;
lane 3) Galα3Galβ4GlcNH₂Galβ4Glcβ1Cer, 4 μg;
lane 4) Galα3(Fucα2)Galβ4GlcNAcβ3Galβ4Glcβ1Cer (B6 type 2 glycosphingolipid), 4 μg;
lane 5) GlcNAcβ3Galβ4GlcNAcβ3Galβ4Glcβ1Cer, 4 μg;
lane 6) Galβ4GlcNAcβ3Galβ4GlcNAcβ3Galβ4Glcβ1Cer, 4 μg;
lane 7) GalNAcβ3Galβ4GlcNAcβ3Galβ4Glcβ1Cer (x₂ glycosphingolipid), 4 μg;
lane 8) NeuAcα3GalNAcβ3Galβ4GlcNAcβ3Galβ4Glcβ1Cer (NeuAc-x₂), 4 μg;
lane 9) Fucα2Galβ4GlcNAcβ3Galβ4Glcβ1Cer (H5 type 2 glycosphingolipid), 4 μg;
lane 10) NeuAcα3Galβ4GlcNAcβ3Galβ4Glcβ1Cer (sialylneolactotetraosylceramide), 4 μg. The sources of the glycosphingolipids are the same as given in Table 1.

FIG. 2. MALDI-TOF mass spectrum of the purified amidation product of GlcAβ3Galβ4GlcNAcβ6GlcNAc. A sample of the product was deposited on a DHB matrix and analyzed in the reflector positive ion mode.

FIG. 3. MALDI-TOF mass spectrum of the purified amidation product of chondroitin tetrasaccharide (Glc[ANH₂]β3GalNAcβ4Glc[A-NH₂]β3GalNAc): m/z 797.2 ([M+Na]⁺) and 813.2 ([M+K]⁺), A-NH₂ indicates the glucuronamides —CO—NH₂

FIG. 4. MALDI-TOF mass spectrum of the purified amidation product of chondroitin hexa-saccharide (Glc[A-NH₂]β3GalNAcβ4Glc[A-NH₂]β3GalNAcβ4Glc[A-NH₂]β3GalNAc): m/Z 1175.2 ([M+Na]⁺) and 1191.2 ([M+K]⁺).

### DETAILED DESCRIPTION OF THE INVENTION

WO 02056893 showed optimal trisaccharide binding sequences for Helicobacter pylori having the oligosaccharide sequence
Gal(A)_{q1}(NAc)ᵣ₁/Glc(A)_{q2}(NAc)ᵣ₂α3/β3Galβ4Glc(NAc)ᵤ
wherein q1, q2, r1, r2, and u are each independently 0 or 1, and wherein lactose based sequences are present in polyvalent form or in high concentration. Also non-reducing-end elongated variants were shown to be effective. Moreover, the studies showed that the oligosaccharide sequence can vary in many positions without losing its H. pylori binding activity.

The present invention allows adding specific modifications to the sequence adding possible modifications of the reducing end side monosaccharide residue and additional variations to the middle and reducing end monosaccharide residues. Furthermore, the present invention represents novel derivatives of uronic acids and method for their preparation. Moreover the present invention is directed to the use of the oligosaccharide backbone in screening assays, in design of combinatorial chemistry libraries and in molecular modeling for design of better analogues or derivatives of the oligosaccharide sequences.

The present invention shows that Galβ4 residue can be replaced in the oligosaccharide sequences by GalNAcβ4. The examples show that GalNAc supports the H. pylori binding activity of lacdiNAc type oligosaccharide sequences such as GalNAcβ4GlcNAc, and chondroitin oligosaccharide sequeces Glc(A-NH₂)β3GalNAcβ4Glc(A-NH₂)β3GalNAc, wherein A-NH₂ indicates the glucuronamides, and GlcAβ3GalNAcβ4GlcAβ3GalNAcβ4GlcAβ3GalNAc. The chondroitin structures show the usefulness of the uronic acid also in the middle or at the reducing end of the oligosaccharide chain. The examples further include a library of uronic acid amide derivatives, from methyl amine to octadecylamine, from which smaller structures were found to be more active than larger structures. Furthermore, the novel binding structures share a common conformation. The putative conformation indicates that there are possible areas of the molecules which can be modified such as position number two of Gal/GalNAcβ4 and reducing end Glc/GlcA/GlcNAc, which both obviously tolerate amine derivative group and hydroxylstructures.

The present invention also shows that for the optimal binding interaction the reducing end monosaccharide of the trisaccharide epitope should be in beta configuration. An example shows strong binding to reductively aminated GlcNAcβ3Galβ4GlcNAcβ6GlcNAc, while corresponding alpha anomer GlcNAcα3Galβ4GlcNAcα6GlcNAc was inactive or very weakly active under the experimental conditions. High binding activity was observed when the reducing end monosaccharide is Gal or GlcNAc. These monosaccharides have similar conformation except for N-acetyl substituent in the place of hydroxyl at carbon 2 and epimeric hydroxyls at carbon 4. GalNAc and Glc are preferred homologs having the same modifications. The preferred specificity for β6 type reducing end terminal was thus determined to be β6Hex(Nac)_{0/1}, wherein Hex is preferably Gal or Glc. Surprisingly, the binding activity was strongly reduced, when the reducing end β6-structure is further elongated by another monosaccharide by β4-linkage (GlcNAcβ3Galβ4GlcNAcβ6Galβ4Glc), therefore it is preferred that there is only one monosaccharide in the reducing end (β6Hex(NAc)_{0/1}-structure).

Preferably the reducing end monosaccharide residue is in open chain derivative form. Preferred open chain forms of the reducing end of the substance include
1. reducing form of the reducing end monosaccharide,
2. reduced reducing end monosaccharide,
3. reduced conjugated reducing end monosaccharide, for example reductively aminated reducing end, and
4. open chain conjugated reducing end such as an oxime of an aminooxy structure, NH₂—O—R where R is the rest of the conjugation agent.
   Reduced reducing ends are in aqueous environment balanced in alpha, beta and open chain forms. For example, the oxime of aminooxy acetic acid is usually in alpha, beta and open chain forms in aqueous solutions.

Furthermore, the present invention is especially directed to favourable β6-linked structures at the reducing end which are cost effective to produce and increase the binding to H. pylori. The disaccharide GlcNAcβ6Gal can be produced effectively by organic synthesis or by enzymatic means and the substrate monosaccharides are relatively cheap compared to GalNAc. GlcNAcβ6GlcNAc can be synthesized even more effectively chemically by traditional methods or by acid reversion chemistry. A suitable bacterial transferase for the synthesis of the substance is known.

Structural Formulas of the Preferred Embodiments

The present invention is directed to the following disaccharide to pentasaccharide oligosaccharide sequences for the treatment of Helicobacter pylori. The present invention is further directed to the oligosaccharides as Helicobacter pylori binding substances for treatment of infectious disease, preferably H. pylori dependent diseases. In a specific embodiment the present invention is directed to the chemically modified or analogous oligosaccharide sequences to obtain lower biological degradation and/higher or more specific activity towards H. pylori and/or other pathogens. Especially analogs comprising glucuronylamide are preferred. The present invention is separately directed to the sequences according to the invention for use as medicine, preferably at least trisaccharide sequences are used. The present invention is further directed to pharmaceutical composition comprising terminal oligosaccharide sequences according to the invention.

In the formulas 1-9 the structural element A indicates the presence or absence of uronic acid form of a monosaccharide residue, furthermore A indicates the presence of derivatized uronic acid, preferably as amides of ammonia, —CO—NH2 or organic amines of the carboxylic acid group at the 6 position. Organic amides include preferably alkylamides, cycloalkylamides, and arylamides, including preferably amides from C₁-C₂₂ amines, more preferably C1-C6 amines, even more preferably from C1-C3 amines and most preferably amides from methylamine or ethylamine. Preferably alkylamines of various sizes are aliphatic 1alkylamine. Preferred arylamide has benzoic acid like benzyl-ring structure, preferred alkylamides include linear 1-amino alkanes. The amides of ammonia or methylamine or ethylamine, and amide from ammonia are more preferred when the molecule comprises uronic acid derivative other than non-reducing end uronic acid derivative or when the same oligosaccharide sequence comprises several uronic acid derivatives. Other uronic acid derivatives include esters.

The preferred structures of the novel Helicobacter pylori binding oligosaccharide sequences comprise the terminal trisaccharide or disaccharide structures according to the Formula 1a
[Hex1(A)_{q1}(NAc)ᵣ₁y3]ₛGal(NAc)ᵣ₂β4Glc(A)_{q2}(NAc)ᵣ₃[β3/6Hex3(NAc)ᵣ₅]_{w}
or when w is 0, the minimal structure according to the Formula 1b
[Hex1(A)_{q1}(NAc)ᵣ₁y3]ₛGal(NAc)ᵣ₂β4Glc(A)_{q2}(NAc)ᵣ₃
wherein q1, q2, r1, r2, r3, r5 and s, and w are each independently 0 or 1, and Hex1, and Hex2 is a hexose structures, preferably galactose (Gal) or glucose (Glc) or mannose (Man), most preferably Gal or Glc, which may be further modified by the A and/or NAC groups; y is either alpha or beta indicating the anomeric structure of the terminal monosaccharide residue with the provisions that when w is 0, then at least one of the integers r2, q1 (when s is also 1), or q2 is 1, preferably at least r2 or q2 is 1 and analogs or derivatives of said oligosaccharide sequence having binding activity to Helicobacter pylori for binding or inhibiting Helicobacter pylori.

Brackets [ ] or { } indicate herein that the structure is either present or absent, ( ) indicates presence or absence of a monosaccharide residue derivatizing structure. Preferably, the reducing end monosaccharide is β-linked, when w is 0. In a preferred embodiment the reducing end monosaccharide structure is an open chain reducing end derivative of the monosaccharide unit. Preferably the structure is not linked to a ceramide, more preferably the structure is not linked to a ceramide comprising a hydroxyl fatty acid.

In a preferred embodiment w is 1 and the reducing end monosaccharide is not glycosidically conjugated to another monosaccharide unit, more preferably the reducing end monosaccharide unit is in an open chain reducing end derivative. In a preferred embodiment A in the Formula is amide, methylamide or ethylamide of the carboxylic acid group of the glucuronic acid residue. One embodiment of the present invention is a substance or a receptor binding to Helicobacter pylori comprising the oligosaccharide sequence according to separately preferred formulas:

Formula 2
[Hex1(A)_{q1}(NAc)ᵣ₁y3]ₛGal(NAc)ᵣ₂β4Glc(A)_{q2}(NAc)ᵣ₃β6Hex3(NAc)ᵣ₅
or
Formula 3
[Hex1(A)_{q1}(NAc)ᵣ₁y3]ₛGal(NAc)ᵣ₂β4Glc(A)_{q2}(NAc)ᵣ₃β3Hex3(NAc)ᵣ₅
or
Formula 4
[Hex1(A)_{q1}(NAc)ᵣ₁y3]ₛGal(NAc)ᵣ₂β4Glc(A)_{q2}(NAc)ᵣ₃β3Gal(NAc)ᵣ₅[β4Glc(A)_{q3}(NAc)ᵣ₆]ᵤ
wherein q1, q2, q3, r1, r2, r3, r5, r6, s, t, and u are each independently 0 or 1, and Hex1, and Hex3 are hexose structures, preferably mannose (Man), galactose (Gal) or glucose (Glc), and most preferably Gal or Glc, which may be further modified by A and/or NAc groups; y is either alpha or beta indicating the anomeric structure of the terminal monosaccharide residue with the provisions that at least one of the integers r2, q2, or q3 is 1 or r5 is 1 when u is 1, preferably r2 is 1 or q2 is 1, and analogs or derivatives of said oligosaccharide sequence having binding activity to Helicobacter pylori for binding or inhibiting Helicobacter pylori.

The reducing end monosaccharide structure can be a conjugated derivative of the monosaccharide residue indicated by the formula, preferably an open chain reducing end derivative of the monosaccharide unit. In a preferred embodiment the reducing end monosaccharide structure is an open chain reducing end derivative of the monosaccharide unit. In another preferred embodiment s is 1.

One embodiment of the present invention is a substance or a receptor binding to Helicobacter pylori comprising the oligosaccharide sequence according to separately preferred formulas:

Formula 5
[Hex1(A)_{q1}(NAc)ᵣ₁y3]ₛGal(NAc)ᵣ₂β4Glc(A)_{q2}(NAc)ᵣ₃β3/6{B}ᵥHex3(NAc)ᵣ₅
wherein q1, q2, r1, r2, r3, r4, r5, s, and v are each independently 0 or 1, and Hex1, Hex2 and Hex3 are hexose structures, preferably mannose (Man), galactose (Gal) or glucose (Glc), and most preferably Gal or Glc, which may be further modified by A and/or NAC groups; B is a branch structure Hex2(NAc)ᵣ₄β3, which can be present or absent, v is 0 or v is 1, only when the structure indicated by t is present (t is 1) in β6-linked form; y is either alpha or beta indicating the anomeric structure of the terminal monosaccharide residue with the provisions that at least one of the integers r2 is 1 or q2 is 1 and that when t=0 then u=1, then either q3=1 and r6 is 1; and analogs or derivatives of said oligosaccharide sequence having binding activity to Helicobacter pylori for binding or inhibiting Helicobacter pylori.

The reducing end monosaccharide structure can be a conjugated derivative of the monosaccharide residue indicated by the formula, preferably an open chain reducing end derivative of the monosaccharide unit. In a preferred embodiment the reducing end monosaccharide structure is an open chain reducing end derivative of the monosaccharide unit. In another preferred embodiment s is 1.

A in the above oligosaccharide sequences indicates uronic acid of the monosaccharide residue or carbon 6 derivative of the monosaccharide residue, most preferably the derivative of carbon 6 is an amide of the uronic acid.

GalNAcβ4GlcNAc (LacdiNAc) and GalNAcβ4Glc (Reduced Chondroitin) Type Structures

Preferred oligosaccharide sequences according to Formula 1a, when r is 1 and q2 is O Formula 6,
[Hex1(A)_{q1}(NAc)ᵣ₁y3]ₛGal(NAc)ᵣ₂β4Glc(NAc)ᵣ₃β[β3/6Hex3(NAc)ᵣ₅]_{w}

The following oligosaccharide sequences are among the preferable Helicobacter pylori binding substances for the uses of the invention.
GalNAcα3GalNAcβ4GlcNAc, GalNAcβ3GalNAcβ4GLcNAc,
GalNAcα3GalNAcβ4GlcNAc, GalNAcβ3GalNAcβ4GLcNAc, Galα3GalNAcβ4GlcNAc,
Galβ3GalNAcβ4GlcNAc Glcα3GalNAcβ4GlcNAc, Glcβ3GalNAcβ4GlcNAc,
GlcAβ3GalNAcβ4GlcNAc, GlcAα3GalNAcβ4GlcNAc, and GalNAcβ4GlcNAc

Preferred carboxylic acid reduced chondroitin type oligosaccharides includes
[Glcβ3]ₛ[GalNAcβ4Glc]ₜ[β3GalNAc]ᵤ
wherein s, t and u are integers so that s is either 0 or 1 and u is either 0 or 1 and t has any value or values (in a mixture) from 1 to 10. Preferably t has any value or values from 1 to 5, more preferably t has values from 1 to 4 and most preferably t has values from 1 to 3. The present invention is directed to the substances as medicines, preferred medicines include substance according to the above structure when s1. Glucuronic acids of natural type chondroitin oligosaccharide sequences can be reduced from carbodiimide conjugate or from methylesters by methods known in the art (see for example WO 0123398). Preferred substances includes carboxylic acid reduced forms of preferred chondroitin oligosaccharides described below, for example
Glcβ3GalNAcβ4Glc,
Glcβ3GalNAcβ4Glcβ3GalNAcβ4Glc, and
Glcβ3GalNAcβ4Glcβ3GalNAcβ4Glcβ3GalNAcβ4Glc
Glcβ3GalNAcβ4Glcβ3GalNAc,
Glcβ3GalNAcβ4Glcβ3GalNAcβ4Glcβ3GalNAc, and
Glcβ3GalNAcβ4Glcβ3GalNAcβ4Glcβ3GalNAcβ4Glcβ3GalNAc

The present invention is directed to optimal receptor size glucuronic acid reduced chondroitin oligosaccharides as substances. The preferred reduced oligosaccharide substance contains at least four, more preferably at least five monosaccharide residues. In another embodiment the preferred oligosaccharide has less than 11 monosaccharide residues, more preferably less than nine monosaccharide residues. The present invention is further directed to composition comprising mixtures of the preferred oligosaccharides, which may further comprise smaller similar oligosaccharides. Preferably the mixtures comprise at least 20%, more preferably at least 50% and most preferably at least 80% and even more preferably at least 90% tetra- to octasacharides.

Examples of preferred terminal acidic and derivative oligosaccharide sequences are
GlcANAcβ3Galβ4GlcNAc,
GlcANAcα3Galβ4GlcNAc,
GlcAβ3Galβ4GlcNAc,
GlcAα3Galβ4GlcNAc,
GlcANAcβ3Galβ4Glc
GlcANAcα3Galβ4Glc,
GlcAβ3Galβ4Glc, and
GlcAα3Galβ4Glc

Preferebly the terminal HNK-1 type sequences are according to the structure
Glc(A)_{q1}(NAc)ᵣ₁β3Galβ4Glc(NAc)ᵣ₃β
wherein Hex is Gal or Glc and q1, r1, and r3, and are 0 or 1 independently.
Preferred Tetrasaccharide Sequences Comprising GlcNAc/Glcβ6/, GlcNAcβ6GlcNAc or GlcNAcβ6GalNAc at the Reducing End

Beside the GalNAcβ4-structures and the uronic acid/uronic acid amide structures the present invention finds β6-linked structures at reducing end very favourable for presentation of the terminal trisaccharide epitopes according to Formula 7
Glc(A)_{q1}(NAc)ᵣ₁β3Galβ4Glc(NAc)ᵣ₃β6Hex(NAc)ᵣ₅
wherein Hex is Gal or Glc and q1, r1, r3, and r5 are 0 or 1 independently.

The reducing end monosaccharide structure can be a conjugated derivative of the monosaccharide residue indicated by the formula, preferably an open chain reducing end derivative of the monosaccharide unit. In a preferred embodiment the reducing end monosaccharide structure is an open chain reducing end derivative of the monosaccharide unit.

In a preferred embodiment either q1 is 0 or r1 is 0. In another separate preferred embodiment r3 is 0, the glucose based structures are preferred for their lower production costs. The reducing end structure support the activity of the glucose comprising molecule. In a preferred embodiment the reducing end monosaccharide is GlcNAc or the reducing end disaccharide is GlcNAcβ6GlcNAc. In a preferred embodiment GlcNAcβ6GalNAc-structures are used because these are analogues of natural O-glycan structures.

More preferably the H. pylori binding sequence is
GlcNAcβ3Galβ4GlcNAcβ6GlcNAc,
Glcβ3Galβ4GlcNAcβ6GlcNAc,
GlcAβ3Galβ4GlcNAcβ6GlcNAc,
GlcNAcβ3Galβ4GlcNAcβ6GalNAc,
Glcβ3Galβ4GlcNAcβ6GalNAc,
GlcAβ3Galβ4GlcNAcβ6GalNAc,
GlcNAcβ3 Galβ4GlcNAcβ6Gal,
Glcβ3Galβ4GlcNAcβ6Gal, or
GlcAβ3Galβ4GlcNAcβ6Gal
GlcNAcβ3Galβ4Glcβ6GlcNAc,
GlcNAcβ3Galβ4Glcβ6GalNAc,
GlcNAcβ3Galβ4Glcβ6Gal
GlcAβ3Galβ4Glcβ6GlcNAc,
GlcAβ3Galβ4Glcβ6GalNAc, or
GlcAβ3Galβ4Glcβ6Gal

And most preferably,
GlcNAcβ3Galβ4GlcNAcβ6GlcNAc, or
GlcAβ3 Galβ4GlcNAcβ6GlcNAc.

Furthermore, GlcA can be derivatized with an amide, most preferably with an amide of methylamide, ethylamide or propylamide or amide formed with ammonium/ammonia, —CO—NH₂.

Preferred pentasaccharide sequences of the invention are
GlcAβ3Galβ4GlcNAcβ3Galβ4Glc and
GlcNAcβ3 Galβ4GlcNAcβ6(Galβ3)GalNAc
   Preferred Natural Chondroitin Type Oligosaccharide Sequences

The present invention is especially directed to specific natural type chondroitin oligosaccharide sequences according to Formula 8
[GlcAβ3]ₛ[GalNAcβ4GlcA]ₜ[β3GalNAc]ᵤ
wherein s, t and U are integers so that s is either 0 or 1 and u is either 0 or 1 and has any value or values (in a mixture) from 1 to 10. Preferably t has any value or values from 1 to 5, more, preferably t has values from 1 to 4 and most, preferably t has values from 1 to 3.

In a more preferred embodiment GlcA in the formula indicates glucuronic acid not oligosaccharides are especially preferred for various uses. These are derivatives of natural structures and are therefore especially preferred for functional foods, food additives, nutritional additives, and animal feeds. The present invention is specifically directed to chondroitin oligosaccharides. Preferably, the chondroitin type oligosaccharide sequence is for use as a medicine for infectious diseases. Chondroitin oligosaccharides is considered also useful for therapheutical uses in foods, feeds, nutrients, self medication products, nutritional additives, food additives, and/or feed additives.

In another preferred embodiment GlcA in the formula and specific structures below represent glucuronic acid which is derivatized to an amide, preferred amides includes amides formed from carboxylic acid group and ammonium/ammonia forming structure —CONH₂, organic amides including alkylamides, cycloalkylamides, arylamides, including preferably amides from C1-C22 amines, more preferably C1-C6 amines, even more preferably from C1-C3 amines and most preferably amides from methylamine or ethylamine. Preferred arylamide has benzoic acid like benzyl-ring structure. Preferred alkylamides include linear 1-amino alkanes.

When s is 1, specifically preferred chondroitin oligosaccharide sequences include oligosaccharides with non-reducing end terminal GlcA due to their high affinity towards H. pylori:
GlcAβ3GalNAcβ4GlcA,
GlcAβ3GalNAcβ4GlcAβ3GalNAcβ4GlcA,
GlcAβ3GalNAcβ4GlcAβ3GalNAcβ4GlcAβ3GalNAcβ4GlcA, and
GlcAβ3GalNAcβ4GlcAβ3GalNAcβ4GlcAβ3GalNAcβ4GlcAβ3GalNAcβ4GlcA

A specifically preferred group of oligosaccharide sequences includes structures comprising non-reducing-end terminal GlcA and reducing end terminal GalNAc. Such oligosaccharides can be produced effectively from natural chondroitin sulphates by desulfation and hyaluronidase digestion (or by hyalurondase digestion and desulphation:
GlcAβ3GalNAcβ4Glcβ3GalNAc,
GlcAβ3GalNAcβ4GlcAβ3GalNAcβ4GlcAβ3GalNAc, and
GlcAβ3GalNAcβ4GlcAβ3GalNAcβ4GlcAβ3GalNAcβ4GlcAβ3GalNAc

Yet another preferred embodiment includes oligosaccharide structures when s is 0, contains terminal GalNAc:
GalNAcβ4GlcA,
GalNAcβ4GlcAβ3GalNAcβ4GlcAβ3GalNAcβ4GlcA, and
GalNAcβ4GlcAβ3GalNAcβ4GlcAβ3GalNAcβ4GlcAβ3GalNAcβ4GlcA
GalNAcβ4GlcAβ3GalNAc,
GalNAcβ4GlcAβ3GalNAcβ4GlcAβ3GalNAc, and
GalNAcβ4GlcAβ3GalNAcβ4GlcAβ3GalNAcβ4GlcAβ3GalNAc

The present invention is in its preferred embodiments directed to the natural type oligosaccharide sequences such as chondroitin oligosaccharides as practically pure single components or as mixtures comprising 2-5 major component comprising more than 90% of the oligosaccharide mixture. The chondroitin sulphate oligosaccharides are preferably devoid of sulphate ester modifications. The invention is further directed to analogous H. pylori binding oligosaccharide sequences which have non-reducing terminal delta uronic acid residue (double bond between C4 and C5), which can be created by lyase type enzymes cleaving the corresponding polysaccharide.

Analogous Hyaluronic Acid Oligosaccharide Sequences

Previously it was demonstrated that H. pylori binds to hyaluronic acid polysaccahride, Present invention allows to determine the binding active oligosaccharide epitopes. The present invention is directed to the hyaluronic acid sequences for preparation medicines for the treatment of infectious diseases, especially due to the presence of H. pylori.

The preferred oligosaccharide sequences may also have the structure according to Formula 9b
[GlcAβ3]ₛ[GlcNAcβ4GlcA]ₜ[β3GlcNAc]ᵤ
and carboxylic acid reduced variants produced as described above for chondroitin oligosaccharides according to Formula 9c
[Glcβ3]ₛ[GlcNAcβ4Glc]ₜ[β3GlcNAc]ᵤ
wherein s, t and u are integers so that s is either 0 or 1 and u is either 0 or 1 and t has any value or values (in a mixture) from 1 to 10. Preferably t has any value or values from 1 to 5, more preferably t has values from 1 to 4 and most preferably t has values from 1 to 3. The invention is further directed to analogous H. pylori binding oligosaccharide sequences which have non-reducing terminal delta uronic acid residue (double bond between C4 and C5), which can be created by lyase type enzymes cleaving the corresponding polysaccharide. The invention is further directed to H. pylori binding analog substances and screening higher affinity variants from the analogs based on formulas 1-8 wherein Gal is replaced by Glc.

The present invention is also directed to the hyaluronic acid derivative substances wherein A is amide as described for other oligosaccharide sequences according to the invention, preferably the glucuronic acid is derivatized to amides of ammonia, methylamine, or ethylamine. The invention is further directed to the substances as medicines.

The present invention is directed to optimal receptor size glucuronic acid reduced hyaluronic acid oligosaccharides as substances. The preferred reduced oligosaccharide substance contains at least four, more preferably at least five monosaccharide residues. In another embodiment the preferred oligosaccharide has less than 11 monosaccharide residues, more preferably less than nine monosaccharide residues. The present invention is further directed to composition comprising mixtures of the preferred oligosaccharides, which may further comprise smaller similar oligosaccharides. Preferably the mixtures comprise at least 20%, more preferably at least 50% and most preferably at least 80% and even more preferably at least 90% tetra- to octasacharides.

Methods to Produce Chondroitin Oligosaccharides with Non-Reducing Terminal GlcA

The present invention is further directed to a method producing chondroitin oligosaccharides from chondroitin sulphates. The method steps involve
1. Removal of sulphates from chondroitin sulphate by chemical hydrolysis. There are several useful methods in the art for example incubating at 80-100 degrees of Celsius in dimethylsulfoxide in the presence of methanol or water.
2. Specifically hydrolyzing glycosidic bonds between GalNAc and GlcA.

The specific hydrolysis is preferably performed by acid hydrolysis, more preferably by a strong carboxylix acid, more preferably the strong carboxyliacid is trifluoroacetic acid. In another preferred embodiment the present invention is directed to specific hydrolysis of the desulphated chondroitin by hyaluronidase enzyme. The method steps can be performed in any order. Preferably, step 1 is performed first. The purification of the product oligosaccharides involves anion exchange chromatography for the separation of the oligosaccharides. More preferably the purification involves anion exchange and gel filtration chromatography (size exclusion chromatography).

The present invention is further directed to methods for the production of amidated glucuronic acid comprising oligosaccharides and monosaccharides from glucuronic acid comprising polysaccharides. Examples of the preferred polysaccharides includes pectin, desulphated chondroitin sulphate and hyaluronic acid as well as bacterial exopolysaccharide comprising glucuronic acid. Alternatively, 6-hydroxyls of a polysaccharide can be specifically oxidized to carboxylic acid groups.

The method involves following steps:
1. Optional oxidation of 6 hydroxyls of a polysaccharide to carboxylic acid groups when the substate does not comprise uronic acid groups, or does contain oxidatable 6-hydroxyl groups.
2. Amidation of glucuronic acid residues of the glucuronic acid comprising polysaccharide. Preferably the amidation is performed from the polysaccharide activated by uronium type amide bond synthesis activator. In another embodiment the carboxylic acid is activated by methyl ester.
3. Hydrolysing the polysaccharide to fragments In separately preferred embodiments the fragments are either oligosaccharides or monosaccharides.
4. optionally isolating oligosaccharide by chromatographic means.

In a preferred embodiment chondroitin or hyaluronic acid polysaccharide is amidated and then hydrolyzed to oligosaccharides according to the invention. Preferably hydrolysis is performed by a carboxylic acid which is more acidic than acetic acid, preferably by trifluoroacetic acid.

Preferred Substances

Present invention is further directed to novel substances which are useful for multiple therapheutical and other uses:
[Hex1(A)_{q1}(NAc)ᵣ₁y3]ₛ₁Gal(NAc)ᵣ₂β4Glc(A)_{q2}(NAc)ᵣ₃
wherein q1, q2, r1, r2, r3, r5 and s, and w are each independently 0 or 1, and Hex1, and Hex2 is a hexose structures, preferably galactose (Gal) or glucose (Glc), which may be further modified by the A and/or NAC groups; y is either alpha or beta indicating the anomeric structure of the terminal monosaccharide residue with the provisions that when s is 0, q2 is 1 and A indicates an amide, when S is 1 both, q1, q2 are 1 with the provision that the molecule does not comprise two β-linked glucuronic acid units, or q1 is 1 and q2 is 0 then y is alpha or r2 is 1, preferably A indicates glucuronic acid amid methylamide or ethylamide.
Characterization of the Overlapping Oligosaccharide Binding Specificties

The present invention describes a family of specific oligosaccharide sequences binding to Helicobacter pylori. The structures of the glycosphingolipids used were characterized by proton NMR and mass spectrometric experiments. Numerous naturally occuring glycosphingolipids have been previously screened (in FI 20010118) by thin-layer overlay assay (Table 1).

In this invention the terms “analog” and “derivative” are defined as follows. According to the present invention it is possible to design structural analogs or derivatives of the Helicobacter pylori binding oligosaccharide sequences. Thus, the invention is also directed to the structural analogs and derivatives of the substances according to the invention. The structural analogs according to the invention are molecules different from original oligosaccharide structures studied and comprise the structural elements important for the binding of Helicobacter pylori to the oligosaccharide sequences. The analogue is such molecule which can not be produced by chemical derivatization reaction in a few steps from the original oligosaccharide backbone while derivatives can be produced by chemical manipulation of the oligosaccharide structures of the invention, derivative can be naturally produced also using derivatized monosaccharide building blocks or by other means. For example derivatives can be easily produced from amino groups after de-N-acetylation of one or several N-acetyl groups of the molecule. Derivatives can also be effectively produced from carboxylic acids groups of uronic acids and even hydroxyl groups can be derivatized for example by ether or ester groups.

Design of Preferred Structural Analogs and Derivatives

The present invention is also specifically directed to the design of structural analogs and derivatives for the Helicobacter pylori binding oligosaccharide sequences. For design of effective structural analogs and derivatives it is important to know the structural element essential for the binding between Helicobacter pylori and the saccharides. The important structural elements are preferably not modified or these are modified by very close mimetic of the important structural element. These elements preferably include the 4-, and 6-hydroxyl groups of the Galβ4 residue in the trisaccharide and disaccharide epitopes. Also the positioning of the linkages between the ring structures is an important structural element. For a high affinity binding the acetamido group or acetamido mimicking group is preferred in the position corresponding to the acetamido group of the reducing end-GlcNAc of the di- or trisaccharide epitopes. Acetamido group mimicking group may be another amide, such as alkylamido, arylamido, secondary amine, preferentially N-ethyl or N-methyl, O-acetyl, or O-alkyl for example O-ethyl or O-methyl. For high affinity binding amide derivatives from carboxylic acid group of the terminal uronic acid and analogues thereof are preferred. The activity of non-modified uronic acid is considered to rise in lower pH.

The structural derivatives according to the invention are oligosaccharide sequences according to the invention modified chemically so that the binding to the Helicobacter pylori is retained or increased. According to the invention it is preferred to derivatize one or several of the hydroxyl or acetamido groups of the oligosaccharide sequences. The invention describes several positions of the molecules which could be changed when preparing the analogs or the derivatives. The hydroxyl or acetamido groups which tolerate at least certain modifications are indicated by R-groups in Formula 9. The present invention is in a preferred embodiment directed for the production of analogues or derivatives according to the Formula 9.

More preferably the analogues are tested for binding or inhibition of Helicobacter pylori and best binding sequences are selected for development of a product. In another embodiment the molecules according to the invention or analogues or derivatives are tested for binding of other microbes or viruses, preferably for binding to toxin A of Clostridium difficile.

Bulky or bulky acidic substituents having size of a monosaccharide residue and other structures, such as monosaccharide residues, are not preferred at least when linked in the position of the C2, C3 or C6-hydroxyls of the Gal-residue Galβ4GlcNAc and on C3-hydroxyl of the non-reducing terminal monosaccharide of the minimal trisaccharide epitopes. Preferably, the analogues designed do not comprise substituents or bulky substituents at these positions.

Methods to produce oligosaccharide analogs for the binding of a lectin are well known. For example numerous analogs of sialyl-Lewis x oligosaccharide hag been produced, representing the active functional groups different scaffold, see page 12090, Sears and Wong 1996. Similarily analogs of heparin oligosaccharides has been produced by Sanofi corporation and sialic acid mimicking inhibitors for the sialidase enzyme, such as accepted medical sialidase inhibitors against influenza by Hofmann-La Roche or Glaxo-Wellcome, by numerous groups.

The present invention is specifically directed to the design of analogues of the oligosaccharide structures according to the invention comprising ring structures analogous to the monosaccharide residues of the oligosaccharide residues. More preferably the analogues are tested for binding or inhibition of Helicobacter pylori and best binding sequences are selected for development of a product. In another embodiment the molecules according to the invention or analogues or derivatives are tested for binding of other microbes or viruses, preferably for binding to toxin A of Clostridium difficile. Preferably the oligosaccharide analog is build on a molecule comprising at least one six- or five-membered ring structure, more preferably the analog contains at least two ring structures comprising 6 or 5 atoms. A preferred analogue type of the oligosaccharide comprise a terminal uronic acid amide or analogue or derivative thereof linked to Gal/GalNAcβ4GlcNAc-saccharide mimicking structure. According to the invention 2 and 4 hydroxyl groups of the terminal monosaccharide residue are not important for binding and 6-hydroxyl can be modified to structures actually increasing the affinity of the molecule, high affinity analogs can be produced when these positions are modified. The data shows that it is possible to design analogs which do not comprise all hydroxyl groups of the terminal monosaccharide residue. The present invention is specifically directed to attaching various organic derivatization molecule such as aromatic or aliphatic cyclic organic residue for the 3-position of terminal Gal/GalNAc production functional analog design. The derivatization may be produced by special linker chemistry allowing lining the cyclic organic residues to 3-position of Gal/GalNAcβ4. The geometry and even length of linking structures may be different from glycosidic bond structures provided that the cyclic organic residue can have at least some of the positive binding interactions of the corresponding terminal monosaccharide residue, especially close to the position of 6-hydroxy/carboxyl/amide of the terminal monosaccharide residue.

In a specific embodiment the analogs are produced from amine group by replacement of hydroxyl group at position 3 of Gal/GalNAcβ4. The present invention is specifically directed to the screening of analogs comprising terminal cyclic molecule on terminal 3-position of Gal/GalNAc in the trisaccharide epitope for binding to Helicobacter pylori. The terminal cyclic molecule is preferably a six membered organic residue, and more preferably it also comprises a carboxylic acid, an amide or alkyl amide structure similar to the structures terminal and/or in the middle hexuronic acids in the formulas according the invention.

Alternatively terminal uronic acid amide or analogue or derivative is 1-3-linked to Gal, which is linked to the GlcNAc mimicking structure. In mimicking structures monosaccharide rings may be replaced by rings such as cyclohexane or cyclopentane, aromatic rings including benzene ring, heterocyclic ring structures may comprise beside oxygen for example nitrogen and sulphur atoms. To lock the active ring conformations the ring structures may be interconnected by tolerated linker groups. Typical mimetic structure may also comprise peptide analog-structures for the oligosaccharide sequence or part of it. The present invention is also directed to the design and/or screening of peptide analogs for the oligosaccharide sequences. Furthermore the present invention is directed for screening of DNA or RNA-based analogues, for example so called aptamers, of the oligosaccharide sequences according to the invention. The effects of the active groups to binding activity are cumulative and lack of one group could be compensated by adding an active residue on the other side of the molecule.

Molecular modelling, preferably by a computer, can be used to produce analog structures for the Helicobacter pylori binding oligosaccharide sequences according to the invention. The results from the molecular modelling of several oligosacharide sequences are given in examples and the same or similar methods, besides NMR and X-ray crystallography methods, can be used to obtain structures for other binding oligosaccharide sequences or analogues or derivatives according to the invention. In a preferred embodiment the analogues or derivatives of the oligosaccharide structures having same or similar conformations with the oligosaccharide structures according to the invention are selected from computer assisted molecular modeling results or screening database or databases containing three dimensional structures of molecules, these methods are referred as computerized screening methods.

The present invention is further directed to the testing oligosaccharide structures and analogues and derivatives thereof which are selected by the computerized screening methods for binding to other pathogenic microbes or viruses or toxins having a binding specificity similar to the binding specificity of Helicobacter pylori with regard to one or several oligosaccharide sequences according to the invention. In a preferred embodiment the oligosaccharide structures and analogues and derivatives selected by the computerized screening methods are tested for binding to toxin A of Clostridium difficile.

The analogue molecules can be synthetically produced or obtained from natural sources. Molecules can also be produced virtually in computers and part of the screening of the active molecules can also be performed in silico. The present invention is also directed to the searching of Helicobacter pylori-binding and/or inhibiting analogues and/or derivatives for the oligosaccharide structures according to the invention by computerized fitting of a carbohydrate structure, analogue or derivative to a carbohydrate binding site on H. pylori.

The Helicobacter pylori-binding oligosacchride sequence, analogues or derivatives thereof are “docked” by methods of molecular modeling to the carbohydrate binding molecule(s) of Helicobacter pylori, most probably to lectins of the bacterium and additional binding interactions are searched. The computerized docking of a three dimensional structure of the oligosaccharide sequence on a three dimensional model of a carbohydrate binding site further helps the design of binding active analogues by allowing determination of binding interactions and positions for possible additional binding interactions. The method is also directed to the comparison of the binding of the oligosaccharide structures and analogues and derivatives thereof by the computerized docking methods.

The present invention is further directed to the testing of oligosaccharide structures and analogues and derivatives thereof by the computerized docking methods for binding to other pathogenic microbes or viruses or toxins having a binding specificity similar to the binding specificity of Helicobacter pylori with regard to one or several oligosaccharide sequences according to the invention. In a preferred embodiment the oligosaccharide structures and analogues and derivatives thereof are tested for binding to toxin A of Clostridium difficile by the computerized docking methods.

It is also noted that the monovalent, oligovalent or polyvalent oligosaccharides can be activated to have higher activity towards lectins by making a derivative of the oligosaccharide by combinatorial chemistry. When a library is created by substituting one or few residues in the oligosaccharide sequence, it can be considered as a derivative library. Alternatively, when the library is created from the analogs of the oligosaccharide sequences described by the invention, it can be considered as an analog library. A combinatorial chemistry library can be built on the oligosaccharide or its precursor or on glycoconjugates according to the invention. For example, oligosaccharides with variable reducing end can be produced by so called carbohydrid technology. The present invention is directed to the design and production of a combinatorial chemistry library, a multide of chemical analogues and/or derivatives of the oligosaccharide structures according to the invention, and testing these for binding or inhibition of Helicobacter pylori. The present invention is further directed to the testing of the combinatorial chemistry library for binding to other pathogenic microbes or viruses or toxins having a binding specificity similar to the binding specificity of Helicobacter pylori with regard to one or several oligosaccharide sequences according to the invention. In a preferred embodiment the combinatorial chemistry library is tested for binding to toxin A of Clostridium difficile.

In a preferred embodiment a combinatorial chemistry library is conjugated to the Helicobacter pylori binding substances described by the invention. In a more preferred embodiment the library comprises at least 6 different molecules. Preferably the combinatorial chemistry modifications are produced by different carboxylic acid group on R₈ or R₉ according to Formula 9. Group to be modified in R₈ may also be an aldehyde or amine or another type of reactive group. Such library is preferred for use o assaying microbial binding to the oligosaccharide sequences according to the invention. Amino acids or collections of organic amides are commercially available, which substances can be used for the synthesis of combinatorial library of uronic acid amides. A high affinity binder could be identified from the combinatorial library for example by using an inhibition assay, in which the library compounds are used to inhibit the bacterial binding to the glycolipids or glycoconjugates described by the invention. Structural analogs and derivatives preferred according to the invention can inhibit the binding of the Helicobacter pylori binding oligosaccharide sequences according to the invention to Helicobacter pylori.

In the following the Helicobacter pylori binding sequence is described as an oligosaccharide sequence. The oligosaccharide sequence defined here can be a part of a natural or synthetic glycoconjugate or a free oligosaccharide or a part of a free oligosaccharide. Such oligosaccharide sequences can be bonded to various monosaccharides or oligosaccharides or polysaccharides on polysaccharide chains, for example, the saccharide sequence is expressed as part of a bacterial polysaccharide. Moreover, numerous natural modifications of monosaccharides are known as exemplified by O-acetyl or sulphated derivatives of the oligosaccharide sequences. In a limited embodiment the oligosaccharide sequence means terminal non-reducing end oligosaccharide sequence which is not modified by any other monosaccharide residue, except optionally at the reducing end. Preferably in broadest sense the term oligosaccharide sequence includes structural analogues and derivatives of the oligosaccharide structures according to the invention, preferably as described by the invention, having same or similar binding activity with regard to the H. pylori. The Helicobacter pylori binding substance defined here can comprise the oligosaccharide sequence described as a natural or synthetic glycoconjugate or part thereof or a corresponding free oligosaccharide or a part of a free oligosaccharide. The Helicobacter pylori binding substance can also comprise a mixture of the Helicobacter pylori binding oligosaccharide sequences.

The Helicobacter pylori binding oligosaccharide sequences can be synthesized enzymatically by glycosyltransferases, or by transglycosylation catalyzed by glycosidase or transglycosidase enzymes (Ernst et al., 2000). Specifities of these enzymes and the use of co-factors can be engineered. Specific modified enzymes can be used to obtain more effective synthesis, for example, glycosynthase is modified to perform transglycosylation only. Organic synthesis of the saccharides and the conjugates is described herein or compounds similar to these are known (Ernst et al., 2000). Saccharide materials can be isolated from natural sources and modified chemically or enzymatically into the Helicobacter pylori binding compounds. Natural oligosaccharides can be isolated from milks of various ruminants. Transgenic organisms, such as cows or microbes, expressing glycosylating enzymes can be used for the production of the saccharides.

The uronic acid monosaccharide residues described in the invention can be obtained by methods known in the art. For example, the hydroxyl of the 6-carbon of N-acetylglucosamine or N-acetylgalactosamines can be chemically oxidized to carboxylic acid. The oxidation can be performed to a properly protected oligosaccharide or monosaccharide or even to a non-protected carbohydrate. In a preferred embodiment a non-protected polymer or oligomer comprising hexoses, N-acetylhexosamines or hexosamines, wherein the linkage between the monosaccharides is not between carbon 6 atoms, is
1. oxidized to corresponding polymer of uronic acid residues, or to polymer comprising monomers of 6-aldehydomonosaccharides
2. optionally derivatized from the carboxylic acid group or 6-aldehydo group, preferentially to an amide or an amine and
3) hydrolysed to the uronic acid monosaccharides or uronic acid derivative monosaccharides.
   Methods to oxidize monosaccharide residues to uronic acids and to hydrolyse amine or uronic acid polymers chemically or enzymatically are well-known in the art. It is especially preferred to use the method for oligomers or polymers of cellulose, starch or other glucans with 1-2 or 1-3 or 1-4 linkages, chitin (GlcNAc polymer) or chitosan (GlcN polymer), which are commercially available in large scale or N-acetylgalactosamine/galactosamine polysaccharides (for example, ones known from a bacterial source) is oxidized to a corresponding 1-4-linked saccharide. This method can also be applied to galactan polymers. Derivatives of uronic acid can be produced also from natural polymers comprising uronic acids such as pectins or glucuronic acid containing bacterial polysaccharides including N-acetylheparin, hyaluronic and chondroitin type animal or bacterial polysaccharides. This method involves
1. derivatization of the carboxylic acid groups of the polysaccharide, preferably by amide bond and
2. hydrolysis of the polysaccharide to the uronic acid monosaccharides or uronic acid derivative monosaccharides.

Chemical and enzymatic methods are also known to oxidize primary alcohol on carbon 6 of the polysaccharide to aldehyde or to carboxylic acid. An aldehyde can be further derivatized, for example, to amine by reductive amination. Preferably terminal Gal or GalNAc is oxidized by a primary alcohol oxidizing enzyme-like galactose oxidase and can then be further derivatized, for example, by amines.

The uronic acid residues can be conjugated to disaccharides or oligosaccharides by standard methods of organic chemistry. Alternatively GlcA can be linked by a glucuronyl transferase transferring a GlcA from UDP-GlcA to terminal Lac(NAc).

Monosaccharide derivatives mimicking N-acetylhexosamines could be produced from a polymer or an oligomer comprising hexosamines or other monosaccharides with free primary amine groups by method involving:
1. derivatization of the amine groups to a secondary or tertiary amine or amide
2. hydrolysing the polymer to corresponding monosaccharides.

Chitosan and oligosaccharides thereof are an example of an amine comprising polymer or oligomer.

In general the method to produce carboxylic acid containing, 6-aldehydo comprising, amine and/or amide comprising monosaccharide/monosaccharides involves following steps
1. optionally introducing an carboxylic acid or 6-aldehydo group to a carbohydrate polymer wherein primary hydroxyl is available for modification
2. derivatization of carboxylic acid groups or 6-aldehydo groups or primary amine groups of the polymer to secondary or tertiary amines or to amides, when step 1 is applied, step 2 is optional.
3. hydrolysis of the polymer to corresponding monosaccharides.

The hydrolysis to monosaccharides may also be partial and produce useful disaccharide or oligosaccharide to produce analog substances. Preferably the hydrolysis produces at least 30% of monosaccharides. Methods to produce the chemical steps are known in the art. For example oxidation of the Polysaccharides to corresponding monosaccharides can be performed as described by Muzzarelli et al 1999 and 2002. These methods are preferred to the use of non-protected monosaccharides, because the protection or reactive reducing ends of the monosaccharides is avoided.

In a preferred embodiment the oligosaccharide sequences comprising GlcAβ3Lac or GlcAβ3LacNAc are effectively synthesised by transglycosylation using a specific glucuronidase such as glucuronidase from bovine liver. It was realized that the enzyme can site-specifically transfer from β1-3 linkage to Galβ4GlcNAc and Galβ4Glc with unexpectedly high yields for a transglycosylation reaction. In general such selectivity and yields close 30% or more are not obtained in transglycosylation reactions.

Another embodiment of the invention is described in Formula 9. The formula describes preferrable derivatives and analogues of the structures according to the formula 1. The formula shows also preferable modifications for producing structural analogues of the sequences.
Formula 9:

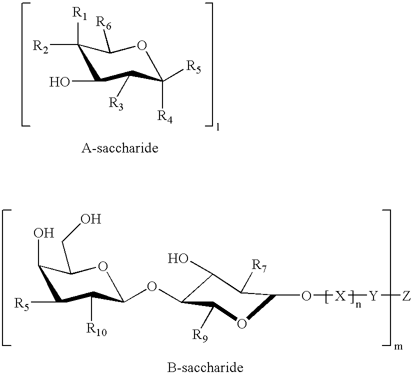

Among the preferable Helicobacter pylori binding substances or mixtures of the substances of the invention and for the uses of the invention are the oligosaccharide structures according to Formula 9, wherein integers 1, m, and n have values m=1, 1 and n are independently 0 or 1, and wherein R₁ is H and R₂ is OH or R₁ is OH and R₂ is H or R₁ is H and R₂ is a monosaccharidyl- or oligosaccharidyl-group preferably a beta glycosidically linked galactosyl group, R₃ is independently —OH or acetamido (—NHCOCH₃) or an acetamido analogous group. R₇ is acetamido (—NHCOCH₃) or an acetamido analogous group. When 1=1, R₄ is —H and R₅ is oxygen linked to bond. R₆ and forms a beta anomeric glycosidic linkage to saccharide B or R₅ is —H and R₄ is oxygen linked to bond R₆ and forms an alpha anomeric glycosidic linkage to saccharide B, when 1=0 R₆ is —OH linked to B. X is monosaccharide or oligosaccharide residue, preferably X is lactosyl-, galactosyl-, poly-N-acetyl-lactosaminyl, or part of an O-glycan or an N-glycan oligosaccharide sequence; Y is a spacer group or a terminal conjugate such as a ceramide lipid moiety or a linkage to Z. Z is an oligovalent or a polyvalent carrier. The binding substance may also be an analog or derivative of said substance according to Formula 9 having binding activity with regard to Helicobacter pylori, e.g., the oxygen linkage (—O—) between position C1 of the B saccharide and saccharide residue X or spacer group Y can be replaced by carbon (—C—), nitrogen (—N—) or sulphur (—S—) linkage.

In Formula 9 R₈ is preferably carboxylic acid amide, such as —CO—NH₂, methylamide or ethyalamide, hydroxymethyl (—CH₂—OH) or a carboxylic acid group or an ester thereof, such as methyl or ethyl ester. The carboxylic acid amide may comprise an alternative linkage to the polyvalent carrier Z comprising an amine such as chitosan or galactosamine polysaccharide or Z comprising a primary amine containing spacer, preferably a hydrophilic spacer. The structure in R₈ can be also a mimicking structure known in the art to ones described above. For example secondary or tertiary amines or amidated secondary amine can be used.

In Formula 9 R₉ is preferably hydroxymethyl or carboxylic acid amide, but it can be used for derivatisations as described for R₈. R₃ is hydroxyl, acetamido or acetamido group mimicking group, such as C₁₋₆ alkylamides, arylamido, secondary amine, preferentially N-ethyl or N-methyl, O-acetyl, or O-alkyl for example O-ethyl or O-methyl. R₇ and R₁₀ are same as R₃ but more preferentially acetamido or acetamido mimicking group. R₂ may also comprise preferentially a six-membered ring structure mimicking Galβ4-terminal.

The bacterium binding substances are preferably represented in clustered form such as by glycolipids on cell membranes, micelles, liposomes, or on solid phases such as TCL-plates used in the assays. The clustered representation with correct spacing creates high affinity binding.

According to the invention it is also possible to use Helicobacter pylori binding epitopes or naturally occurring, or a synthetically produced analogue or derivative thereof having a similar or better binding activity with regard to Helicobacter pylori. It is also possible to use a substance containing the bacterium binding substance such as a receptor active ganglioside described in the invention or an analogue or derivative thereof having a similar or better binding activity with regard to Helicobacter pylori. The bacterium binding substance may be a glycosidically linked terminal epitope of an oligosaccharide chain. Alternatively the bacterium binding epitope may be a branch of an oligosaccharide chain, preferably a polylactosamine chain.

The Helicobacter pylori binding substance may be conjugated to an antibiotic substance, preferably a penicillin type antibiotic. The Helicobacter pylori binding substance targets the antibiotic to Helicobacter pylori. Such conjugate is beneficial in treatment because a lower amount of antibiotic is needed for treatment or therapy against Helicobacter pylori, which leads to lower side effect of the antibiotic. The antibiotic part of the conjugate is aimed at killing or weaken the bacteria, but the conjugate may also have an antiadhesive effect as described below.

The bacterium binding substances, preferably in oligovalent or clustered form, can be used to treat a disease or condition caused by the presence of the Helicobacter pylori. This is done by using the Helicobacter pylori binding substances for anti-adhesion, i.e. to inhibit the binding of Helicobacter pylori to the receptor epitopes of the target cells or tissues. When the Helicobacter pylori binding substance or pharmaceutical composition is administered it will compete with receptor glycoconjugates on the target cells for the binding of the bacteria. Some or all of the bacteria will then be bound to the Helicobacter pylori binding substance instead of the receptor on the target cells or tissues. The bacteria bound to the Helicobacter pylori binding substances are then removed from the patient (for example by the fluid flow in the gastrointestinal tract), resulting in reduced effects of the bacteria on the health of the patient. Preferably the substance used is a soluble composition comprising the Helicobacter pylori binding substances. The substance can be attached to a carrier substance which is preferably not a protein. When using a carrier molecule several molecules of the Helicobacter pylori binding substance can be attached to one carrier and inhibitory efficiency is improved.

The target cells are primarily epithelial cells of the target tissue, especially the gastrointestinal tract, other potential target tissues are for example liver and pancreas. Glycosylation of the target tissue may change because of infection by a pathogen (Karlsson et al., 2000). Target cells may also be malignant, transformed or cancer/tumour cells in the target tissue. Transformed cells and tissues express altered types of glycosylation and may provide receptors to bacteria. Binding of lectins or saccharides (carbohydrate-carbohydrate interaction) to saccharides on glycoprotein or glycolipid receptors can activate cells, in case of cancer/malignant cells this may be lead to growth or metastasis of the cancer. Several of the oligosaccharide epitopes described herein, such as GlcNAcβ3Galβ4GlcNAc (Hu, J. et al., 1994), Galα3Galβ4GlcNAc (Castronovo et al., 1989), and neutral and sialylated polylactosamines from malignant cells (Stroud et al., 1996), have been reported to be cancer-associated or cancer antigens. Oligosaccharide chains containing substances have also been described from lymphocytes (Vivier et al., 1993). Helicobacter pylori is associated with gastric lymphoma. The substances of the invention can be used to prevent binding of Helicobacter pylori to premalignant or malignant cells and activation of cancer development or metastasis. Inhibition of the binding may cure gastric cancer, especially lymphoma. The Helicobacter pylori binding oligosaccharide sequence has been reported in the structure GlcNAcβ3Galβ4GlcNAcβ6GalNAc from human gastric mucins. This mucin epitope and similar O-glycan glycoforms are most probably natural high affinity receptors for Helicobacter pylori in human stomach. This was also indicated by high affinity binding of an analogous sequence GlcNAcβ3Galβ4GlcNAcβ6GlcNAc as neoglycolipid to Helicobacter pylori and that the sequence GlcNAcβ3Galβ4GlcNAcβ6Gal has also some binding activity towards Helicobacter pylori in the same assay. Therefore the preferred oligosaccharide sequences includes O-glycans and analogues of O-glycan sequences such as GlcNAcβ3Galβ4GlcNAcβ6GlcNAc/GalNAc/Gal, GlcNAcβ3Galβ4GlcNAcβ6GlcNAc/GalNAc/GalαSer/Thr, GlcNAcβ3Galβ4GlcNAcβ6(Gal/GlcNAcβ3)GlcNAc/GalNAc/GalαSer/Thr and glycopeptides and glycopeptide analogs comprising the O-glycan sequences. Even sequences lacking the non-reducing end GlcNAc may have some activity. Based on this all the other Helicobacter pylori binding oligosaccharide sequences (OS) and especially the trisaccharide epitopes are also especially preferred when linked from the reducing end to form structures OSβ6Gal(NAc)₀₋₁ or OSβ6Glc(NAc)₀₋₁ or OSβ6Gal(NAc)₀₋₁αSer/Thr or OSβ6Glc(NAc)₀₋₁αSer/Thr. The Ser or Thr-compounds or analogue thereof or the reducing oligosaccharides are also preferred when linked to polyvalent carrier. The reducing oligosaccharides can be reductively linked to the polyvalent carrier.

Target cells also includes blood cells, especially leukocytes. It is known that Helicobacter pylori strains associated with peptic ulcer, as the strain mainly used here, stimulates an inflammatory response from granulocytes, even when the bacteria are nonopsonized (Rautelin et al., 1994a,b). The initial event in the phagocytosis of the bacterium most likely involves specific lectin-like interactions resulting in the agglutination of the granulocytes (Ofek and Sharon, 1988). Subsequent to the phagocytotic event oxidative burst reactions occur which may be of consequence for the pathogenesis of Helicobacter pylori-associated diseases (Babior, 1978). Several sialylated and non-acid glycosphingolipids having repeating N-acetyllactosamine units have been isolated and characterized from granulocytes (Fukuda et al., 1985; Stroud et al., 1996) and may thus act as potential receptors for Helicobacter pylori on the white blood cell surface. Furthermore, also the X₂ glycosphingolipid has been isolated from the same source (Teneberg, S., unpublished). The present invention confirms the presence of receptor saccharides on human erythrocytes and granulocytes which can be recognized by an N-acetyllactosamine specific lectin and by a monoclonal antibody (x₂, GalNAcβ3Galβ4GlcNAc-). The Helicobacter pylori binding substances can be useful to inhibit the binding of leukocytes to Helicobacter pylori and in prevention of the oxidative burst and/or inflammation following the activation of leukocytes.

It is known that Helicobacter pylori can bind several kinds of oligosaccharide sequences. Some of the binding by specific strains may represent more symbiotic interactions which do not lead to cancer or severe conditions. The present data about binding to cancer-type saccharide epitopes indicates that the Helicobacter pylori binding substance can prevent more pathologic interactions, in doing this it may leave some of the less pathogenic Helicobacter pylori bacteria/strains binding to other receptor structures. Therefore total removal of the bacteria may not be necessary for the prevention of the diseases related to Helicobacter pylori. The less pathogenic bacteria may even have a probiotic effect in the prevention of more pathogenic strains of Helicobacter pylori.

It is also realized that Helicobacter pylori contains large polylactosamine oligosaccharides on its surface which at least in some strains contains non-fucosylated epitopes which can be bound by the bacterium as described by the invention. The substance described herein can also prevent the binding between Helicobacter pylori bacteria and that way inhibit bacteria for example in process of colonization.

According to the invention it is possible to incorporate the Helicobacter pylori binding substance, optionally with a carrier, in a pharmaceutical composition, which is suitable for the treatment of a condition due to the presence of Helicobacter pylori in a patient or to use the Helicobacter pylori binding substance in a method for treatment of such conditions. Examples of conditions treatable according to the invention are chronic superficial gastritis, gastric ulcer, duodenal ulcer, non-Hodgkin lymphoma in stomach, gastric adenocarcinoma, and certain pancreatic skin, liver, or heart diseases, sudden infant death syndrome, autoimmune diseases including autoimmune gastritis and pernicious anaemia and non-steroid anti-inflammatory drug (NSAID) related gastric disease, all, at least partially, caused by the Helicobacter pylori infection.

The pharmaceutical composition containing the Helicobacter pylori binding substance may also comprise other substances, such as an inert vehicle, or pharmaceutically acceptable carriers, preservatives etc, which are well known to persons skilled in the art. The Helicobacter pylori binding substance can be administered together with other drugs such as antibiotics used against Helicobacter pylori.

The Helicobacter pylori binding substance or pharmaceutical composition containing such substance may be administered in any suitable way, although an oral administration is preferred.

The term “treatment” used herein relates both to treatment in order to cure or alleviate a disease or a condition, and to treatment in order to prevent the development of a disease or a condition. The treatment may be either performed in a acute or in a chronic way.

The term “patient” or “subject”, as used herein, relates to any human or non-human mammal in need of treatment according to the invention.

It is also possible to use the Helicobacter pylori binding substance to identify one or more adhesins by screening for proteins or carbohydrates (by carbohydrate-carbohydrate interactions) that bind to the Helicobacter pylori binding substance. The carbohydrate binding protein may be a lectin or a carbohydrate binding enzyme. The screening can be done for example by affinity chromatography or affinity cross linking methods (liver et al., 1998).

Furthermore, it is possible to use substances specifically binding or inactivating the Helicobacter pylori binding substances present on human tissues and thus prevent the binding of Helicobacter pylori. Examples of such substances include plant lectins such as Erythrina cristagalli and Erythrina corallodendron (Teneberg et al., 1994). When used in humans, the binding-substance should be suitable for such use such as a humanized antibody or a recombinant glycosidase of human origin which is non-immunogenic and capable of cleaving the terminal monosaccharide residue/residues from the Helicobacter pylori binding substances. However, in the gastrointestinal tract, many naturally occuring lectins and glycosidases originating for example from food are tolerated.

Furthermore, it is possible to use the Helicobacter pylori binding substance as part of a nutritional composition including food- and feedstuff. It is preferred to use the Helicobacter pylori binding substance as a part of so called functional or functionalized food. The said functional food has a positive effect on the person's or animal's health by inhibiting or preventing the binding of Helicobacter pylori to target cells or tissues. The Helicobacter pylori binding substance can be a part of a defined food or functional food composition. The functional food can contain other acceptable food ingredients accepted by authorities such as Food and Drug Administration in the USA. The Helicobacter pylori binding substance can also be used as a nutritional additive, preferably as a food or a beverage additive to produce a functional food or a functional beverage. The food or food additive can also be produced by having, e.g., a domestic animal such as a cow or other animal produce the Helicobacter pylori binding substance in larger amounts naturally in its milk. This can be accomplished by having the animal overexpress suitable glycosyltransferases in its milk. A specific strain or species of a domestic animal can be chosen and bred for larger production of the Helicobacter pylori binding substance. The Helicobacter pylori binding substance for a nutritional composition or nutritional additive can also be produced by a micro-organisms such as a bacteria or a yeast.

It is especially useful to have the Helicobacter pylori binding substance as part of a food for an infant, preferably as a part of an infant formula. Many infants are fed by special formulas in replacement of natural human milk. The formulas may lack the special lactose based oligosaccharides of human milk, especially the elongated ones such as lacto-N-neotetraose, Galβ4GlcNAcβ3Galβ4Glc, and its derivatives. The natural type oligosaccharide derived from desulphated chondroitin, and hyaluronic acid are preferred for infant formulas, other functional foods and food additive. Also the carboxylic acid reduced hyaluronic acid and chondroitin oligosaccharides according to the invention are considered useful for use in infant formulas as close analogs of the natural type polylactosamines and lactose based milk oligosaccharides, the chondroitin saccharides has also homology with core 1 structures of O-linked glycans, moreover disaccharide GalNAcβ4Glc is a component of bovine milk. For example following oligosaccharides are preferred for infant foods, other functional foods, feeds and nutritional additives:
GalNAcβ4Glc
Glcβ3GalNAcβ4Glc,
Glcβ3GalNAcβ4Glcβ3GalNAcβ4Glc,
Glcβ3GalNAcβ4Glcβ3GalNAcβ4Glcβ3GalNAcβ4Glc,
Glcβ3GalNAcβ4Glcβ3GalNAc,
Glcβ3GalNAcβ4Glcβ3GalNAcβ4Glcβ3 GalNAc,
Glcβ3GalNAcβ4Glcβ3GalNAcβ4Glcβ3GalNAc,
Glcβ3GlcNAcβ4Glcβ3GlcNAc,
Glcβ3GlcNAcβ4Glcβ3GlcNAcβ4Glcβ3GlcNAc,
Glcβ3GlcNAc 4Glcβ3GlcNAcβ4Glcβ3GlcNAcβ4Glcβ3GlcNAc,
Glcβ3GlcNAcβ4Glc, Glcβ3GlcNAcβ4Glcβ3GlcNAcβ4Glc,
Glcβ3GlcNAcβ4Glcβ3GlcNAcβ4Glcβ3GlcNAcβ4Glc.

These can be considered as safe additives or ingredients in an infant food. Helicobacter pylori is especially infective with regard to infants or young children, and considering the diseases it may later cause it is reasonable to prevent the infection. Helicobacter pylori is also known to cause sudden infant death syndrome, but the strong antiobiotic treatments used to eradicate the bacterium may be especially unsuitable for young children or infants.

Furthermore, it is possible to use the Helicobacter pylori binding substance in the diagnosis of a condition caused by an Helicobacter pylori infection. Diagnostic uses also include the use of the Helicobacter pylori binding substance for typing of Helicobacter pylori. When the substance is used for diagnosis or typing, it may be included in, e.g., a probe or a test stick, optionally constituting a part of a test kit. When this probe or test stick is brought into contact with a sample containing Helicobacter pylori, the bacteria will bind the probe or test stick and can be thus removed from the sample and further analyzed.

The results also show that the non-reducing end terminal monosaccharide residue in the preferred trisaccharide sequences of the invention can contain a carboxylic acid group on the carbon 6 (terminal monosaccahride residue is a uronic acid, HexA or HexANAc, wherein Hex is Gal or Glc) or a derivative of the carbon 6 of the HexA(NAc) residue or a derivative of the carbon 6 of the corresponding Hex(NAc) residue. Such terminal residues includes preferably β3-linked glucuronic acid and more preferably 6-amides such as methylamide thereof. Therefore analogs and derivatives of the sequence can be produced by changing or derivatising the terminal 6-position of the trisaccharide epitopes.

Preferred Helicobacter pylori Binding Substances

The oligosaccharide sequences according to the invention were found to be unexpectedly effective binders when presented on thin layer surface. This method allows polyvalent presentation of the glycolipid sequences. The surprisingly high activity of the polyvalent presentation of the oligosaccharide sequences makes polyvalency a preferred way to represent the oligosaccharide sequences of the invention.

The glycolipid structures are naturally presented in a polyvalent form on cellular membranes. This type of representation can be mimicked by the solid phase assay described below or by making liposomes of glycolipids or neoglycolipids.

The present novel neoglycolipids produced by reductive amination of hydrophobic hexadecylaniline were able to provide effective presentation of the oligosaccharides. Most previously known neoglycolipid conjugates used for binding of bacteria have contained a negatively charged groups, such as phosphor ester of phosphadityl ethanolamine neoglycolipids. Problems of such compounds are negative charge of the substance and natural biological binding involving the phospholipid structure. Negatively charged molecules are known to be involved in numerous non-specific bindings with proteins and other biological substances. Moreover, many of these structures are labile and can be enzymatically or chemically degraded. The present invention is directed to the non-acidic conjugates of oligosaccharide sequences meaning that the oligosaccharide sequences are linked to non-acidic chemical structures. Preferably, the non-acidic conjugates are neutral meaning that the oligosaccharide sequences are linked to neutral, non-charged, chemical structures. The preferred conjugates according to the invention are polyvalent substances.

In the previous art bioactive oligosaccharide sequences are often linked to carrier structures by reducing a part of the receptor active oligosaccharide structure. Hydrophobic spacers containing alkyl chains (—CH₂—)ₙ and/or benzyl rings have been used. However, hydrophobic structures are in general known to be involved in non-specific interactions with proteins and other bioactive molecules.

The neoglycolipid data of the examples below show that under the experimental conditions used in the assay the hexadecylaniline parts of the neoglycolipid compounds do not cause non-specific binding for the studied bacterium. In the neoglycolipids the hexadecylaniline part of the conjugate forms probably a lipid layer like structure and is not available for the binding. The invention shows that reducing a monosaccharide residue belonging to the binding epitope may destroy the binding. It was further realized that a reduced monosaccharide can be used as a hydrophilic spacer to link a receptor epitope and a polyvalent presentation structure. According to the invention it is preferred to link the bioactive oligosaccharide via a hydrophilic spacer to a polyvalent or multivalent carrier molecule to form a polyvalent or oligovalent/multivalent structure. All polyvalent (comprising more than 10 oligosaccharide residues) and oligovalent/multivalent structures (comprising 2-10 oligosaccharide residues) are referred here as polyvalent structures, though depending on the application oligovalent/multivalent constructs can be more preferred than larger polyvalent structures. The hydrophilic spacer group comprises preferably at least one hydroxyl group. More preferably the spacer comprises at least two hydroxyl groups and most preferably the spacer comprises at least three hydroxyl groups.

According to the invention the hydrophilic spacer group is preferably a flexible chain comprising one or several —CHOH— groups and/or an amide side chain such as an acetamido —NHCOCH₃ or an alkylamido. The hydroxyl groups and/or the acetamido group also protects the spacer from enzymatic hydrolysis in vivo. The term flexible means that the spacer comprises flexible bonds and do not form a ring structure without flexibility. A reduced monosaccharide residues such as ones formed by reductive amination in the present invention are examples of flexible hydrophilic spacers. The flexible hydrophilic spacer is optimal for avoiding non-specific binding of neoglycolipid or polyvalent conjugates. This is essential optimal activity in bioassays and for bioactivity of pharmaceuticals or functional foods, for example.

A general formula for a conjugate with a flexible hydrophilic linker has the following Formula 10:
[OS—O—(X)ₙ-L₁-CH(H/{CH₁₋₂OH}ₚ₁)—{CH₁OH}ₚ₂—{CH(NH—R)}ₚ₃—{CH₁OH}ₚ₄-L₂]ₘ-Z
wherein L₁ and L₂ are linking groups comprising independently oxygen, nitrogen, sulphur or carbon linkage atom or two linking atoms of the group forming linkages such as —O—, —S—, —CH₂—, —N—, —N(COCH3)-, amide groups —CO—NH— or —NH—CO— or —N—N-(hydrazine derivative) or amino oxy-linkages —O—N— and —N—O—. L1 is linkage from carbon 1 of the reducing end monosaccharide of X or when n=0, L1 replaces —O— and links directly from the reducing end C1 of OS.

p1, p2, p3, and p4 are independently integers from 0-7, with the proviso that at least one of p1, p2, p3, and p4 is at least 1. CH₁₋₂OH in the branching term {CH₁₋₂OH}ₚ₁ means that the chain terminating group is CH₂OH and when the p1 is more than 1 there is secondary alcohol groups —CHOH— linking the terminating group to the rest of the spacer. R is preferably acetyl group (—COCH₃) or R is an alternative linkage to Z and then L₂ is one or two atom chain terminating group, in another embodiment R is an analog forming group comprising C₁₋₄ acyl group (preferably hydrophilic such as hydroxy alkyl) comprising amido structure or H or C₁₋₄ alkyl forming an amine. And m>1 and Z is polyvalent carrier. OS and X are defined in Formula 9.

In a preferred embodiment of the invention p₁ is 0. The invention shows that open chain forms of a monosaccharide are effective H. pylori binding structures at the reducing end. These structures can mimic ceramides with hydroxylfattyacid. In a preferred embodiment of the invention the hydrophilic linker according to Formula 10 comprise a hydroxyl group at the same distance from the reducing end oligosaccharide sequence as the corresponding hydroxyl group is on ceramide from the reducing end oligosaccharide in natural glycosphingolipids with hydroxylfattyacid.

Preferred polyvalent structures comprising a flexible hydrophilic spacer according to formula 2 include Helicobacter pylori biding oligosaccharide sequence (OS) β1-3 linked to Galβ4Glc(red)-Z, and OSβ6GlcNAc(red)-Z and OSβ6GalNAc(red)-Z, where “(red)” means the amine linkage structure formed by reductive amination from the reducing end monosaccharides and an amine group of the polyvalent carrier Z.

In the present invention the oligosaccharide group is preferably linked in a polyvalent or an oligovalent form to a carrier which is not a protein or peptide to avoid antigenicity and possible allergic reactions, preferably the backbone is a natural non-antigenic polysaccharide.

When the binding activities of glycolipids and neoglycolipids were compared, the sequences with Galα3Galβ-were found to have lower activity in the polyvalent presentation on thin layer plate. The sequences with terminal Galβ4GlcNAc-sequence were also weaker. Therefore the optimal polyvalent non-acidic substance according to the invention comprises a terminal oligosaccharide sequence
Gal(A)_{q1}(NAc)ᵣ₁/Glc(A)_{q2}(NAc)ᵣ₂α3/β3Galβ4Glc(NAc)ᵤ
wherein q1, q2, r1, r2, and u are each independently 0 or 1,
with the proviso that when both q1 and r1 are 0, then the non-reducing end terminal monosaccharide residue is not Gala. More preferably u=0 and
most preferably the oligosaccharide sequence presented in polyvalent form is
   GalNAc/Glc(NAc)ᵣ₂α3/β3Galβ4GlcNAc
   wherein r2 is independently 0 or 1 and an analog or derivative thereof.

Following oligosaccharide sequences are especially preferred. These represent structures, which have not been described from human or animal tissues:
with the proviso that when the oligosaccharide sequence contains β3 linkage, q and r are 1 or 0; or GalA(NAc)ᵣα3/β3Galβ4Glc(NAc)ᵤ.

The novelty of the above oligosaccharide sequences makes them especially preferred. There are no known glycosidases cleaving such sequences. Therefore, the sequences are especially stable and preferred under biological conditions. The natural type of the sequences described by the invention can be cleaved by glycosidase enzymes which reduces usefulness of these especially when used in human and animal body. Glycosidase enzymes cleaving the sequences are known to be active in human gastrointestinal tract. Several glycosidases such as N-acetylhexosaminidases or galactosidases has been described as digestive enzyme and are also present in food stuffs.

It is realized that the novel substances according to the invention are also useful for inhibiting toxin A of Clostridium difficile S. Teneberg et al 1996. The binding profile of the toxin A with older substances is very similar to specificity of Helicobacter pylori described here. Thus, the Helicobacter pylori binding substances may be used for the treatment, for example, Clostridium difficile dependent diarrhea.

Ex Vivo Uses of the Present Invention

It is realized that the present invention can be used for inhibition of pathogens especially Helicobacter pylori ex vivo and such method have use in disinfection and preservation type applications.

Glycolipid and carbohydrate nomenclature is according to recommendations by the IUPAC-IUB Commission on Biochemical Nomenclature (Carbohydrate Res. 1998, 312, 167; Carbohydrate Res. 1997, 297, 1; Eur. J. Biochem. 1998, 257, 29).

It is assumed that Gal, Glc, GlcNAc, and Neu5Ac are of the D-configuration, Fuc of the L-configuration, and all the monosaccharide units in the pyranose form. Glucosamine is referred as GlcN or GlcNH₂ and galactosamine as GalN or GalNH₂. Glycosidic linkages are shown partly in shorter and partly in longer nomenclature, the linkages of the Neu5Ac-residues α3 and α6 mean the same as α2-3 and α2-6, respectively, and with other monosaccharide residues α1-3, β1-3, β1-4, and β1-6 can be shortened as α3, β3, β4, and β6, respectively. Lactosamine refers to N-acetyllactosamine, Galβ4GlcNAc, and sialic acid is N-acetylneuraminic acid (Neu5Ac) or N-glycolylneuraminic acid (Neu5Gc) or any other natural sialic acid. Term glycan means here broadly oligosaccharide or polysaccharide chains present in human or animal glycoconjugates, especially on glycolipids or glycoproteins. In the shorthand nomenclature for fatty acids and bases, the number before the colon refers to the carbon chain length and the number after the colon gives the total number of double bonds in the hydrocarbon chain. Abbreviation GSL refers to glycosphingolipid. Abbreviations or short names or symbols of glycosphingolipids are given in the text and in Tables 1 and 2. Helicobacter pylori refers also to the bacteria similar to Helicobacter pylori.

In the present invention hex(NAc)-uronic acid and their derivatives and residues are indicated as follows: GlcA is glucuronic acid and derivatives of carbon 6 of glucose or glucuronic acid, GalA is galacturonic acid and derivatives of carbon 6 of galactose or galacturonic acid, GlcANAc is N-acetylglucuronic acid and derivatives of carbon 6 of N-acetylglucosamine or is N-acetylglucosamine uronic acid and GalANAc is N-acetylgalactosamine uronic acid and derivatives of carbon 6 of N-acetylgalactosamine or N-acetylgalactosamine uronic acid.

The expression “terminal oligosaccharide sequence” indicates that the oligosaccharide is not substituted to the non-reducing end terminal residue by another monosaccharide residue.

The term “α3/β3” indicates that the adjacent residues in an oligosaccharide sequence can be either α3- or β3-linked to each other.

The present invention is further illustrated by the following examples, which in no way are intended to limit the scope of the invention:

### EXAMPLES

### Example 1

### General Materials and Methods

Materials—TLC silica gel 60 (aluminum) plates were from Merck (Darmstadt, Germany). Ham's F12 medium from Gibco (U.K.), ³⁵S-methionine from Amersham (U.K.) and FCS (fetal calf serum) was from Sera-Lab (England). The clinical isolates of Helicobacter pylori (strains 002 and 032) obtained from patients with gastritis and duodenal ulcer, respectively, were a generous gift from Dr. D. Danielsson, Örebro Medical Center, Sweden. Type strain 17875 was from Culture Collection, University of Göteborg (CCUG).

Glycosphingolipids. The pure glycosphingolipids of the experiment shown in FIGS. 7A and 7B were prepared from total acid or non-acid fractions from the sources listed in Table 1 as described in (Karlsson, 1987). In general, individual glycosphingolipids were obtained by acetylation (Handa, 1963) of the total glycosphingolipid fractions and separated by repeated silicic acid column chromatography, and subsequently characterized structurally by spectrometry (Samuelsson et al., 1990), NMR (Falk et al., 1979a,b,c; Koerner Jr et al., 1983) and degradative procedures (Yang and Hakomori, 1971; Stellner et al., 1973). Glycolipids derived from rabbit thymys are described below.

De-N-acylation. Conversion of the acetamido moiety of GlcNAc/GalNAc residues into an amine was accomplished by treating various glycosphingolipids with anhydrous hydrazine as described previously (Ångström et al., 1998).

Bacterial growth. The Helicobacter pylori strains were stored at −80° C. in tryptic soy broth containing 15% glycerol (by volume). The bacteria were initially cultured on GAB-CAMP agar (Soltesz et al., 1988) under humid (98%) microaerophilic conditions (O₂: 5-7%, CO₂: 8-10% and N₂: 83-87%) at 37° C. for 48-72 h. For labeling colonies were inoculated on GAB-CAMP agar, except for the results presented in FIGS. 1A and 1B where Brucella agar (Difco, Detroit, Mich.) was used instead, and 50 μCi ³⁵S-methionine (Amersham, U.K.), diluted in 0.5 ml phosphate-buffered saline (PBS), pH 7.3, was sprinkled over the plates. After incubation for 12-24 h at 37° C. under microaerophilic conditions, the cells were scraped off, washed three times with PBS, and resuspended to 1×10⁸ CFU/ml in PBS. Alternatively, colonies were inoculated (1×10⁵ CFU/ml) in Ham's F12 (Gibco BRL, U.K.), supplemented with 10% heat-inactivated fetal calf serum (Sera-Lab). For labeling, 50 μCi ³⁵S-methionine per 10 ml medium was added, and incubated with shaking under microaerophilic conditions for 24 h. Bacterial cells were harvested by centrifugation, and purity of the cultures and a low content of coccoid forms was ensured by phase-contrast microscopy. After two washes with PBS, the cells were resuspended to 1×10⁸ CFU/ml in PBS. Both labeling procedures resulted in suspensions with specific activities of approximately 1 cpm per 100 Helicobacter pylori organisms.

TLC bacterial overlay assay. Thin-layer chromatography was performed on glass- or aluminum-backed silica gel 60 HPTLC plates (Merck, Darmstadt, Germany) using chloroform/methanol/water 60:35:8 (by volume) as solvent system. Chemical detection was accomplished by anisaldehyde staining (Waldi, 1962). The bacterial overlay assay was performed as described previously (Hansson et al., 1985). Glycosphingolipids (1-4 μg/lane, or as indicated in the figure legend) were chromatographed on aluminum-backed silica gel plates and thereafter treated with 0.3-0.5% polyisobutylmethacrylate in diethylether/n-hexane 1:3 (by volume) for 1 min, dried and subsequently soaked in PBS containing 2% bovine serum albumin and 0.1% Tween 20 for 2 h. A suspension of radio-labeled bacteria (diluted in PBS to 1×10⁸ CFU/ml and 1-5×10⁶ cpm/ml) was sprinkled over the chromatograms and incubated for 2 h followed by repeated rinsings with PBS. After drying the chromatograms were exposed to XAR-5 X-ray films (Eastman Kodak Co., Rochester, N.Y., USA) for 12-72 h.

Molecular modeling was performed as described in the priority application and in Teneberg et al-96.

Synthesis of oligosaccharides. The oligosaccharide GlcNAcβ3Galβ4GlcNAc was synthesised from Galβ4GlcNAc (Sigma, St. Louis, USA), GalNAcβ4GlcNAcβ3Galβ4GlcNAc was synthesised from the trisaccharide by transferring GalNAc from vast excess of UDP-GalNAc (Sigma by large amounts of β4Galactosyltransferase (bovine milk, Calbiochem., CA, USA) and GlcNAcβ3Galβ4GlcNAcβ6GlcNAc, GlcNAcβ3Galβ4GlcNAcβ6Galβ4Glc, GlcNAcβ3Galβ4GlcNAcα6GlcNAc, GlcNAcβ3 Galβ4GlcNAcβ3Man were synthesised from Galβ4GlcNAcβ6GlcNAc, Galβ4GlcNAcβ6Galβ4Glc, Galβ4GlcNAcα6GlcNAc, and Galβ4GlcNAcβ3Man, respectively, by incubating the acceptor saccharide with human serum β3-N-acetylglucosaminyltransferase and UDP-GlcNAc in presence of 8 mM MnCl₂ and 0.2 mg/ml ATP at 37 degree of Celsius for 5 days in 50 mM TRIS-HCl pH 7.5. Galβ4GlcNAcβ6GlcNAc, Galβ4GlcNAcβ6Galβ4Glc, Galβ4GlcNAcα6GlcNAc, and Galβ4GlcNAcβ3Man were obtained from GlcNAcβ6GlcNAc (Sigma, St Louis, USA), GlcNAcβ6Galβ4Glc (Sigma), GlcNAcα6GlcNAc (HCl vapor catalyzed acid reversion reaction for solid GlcNAc (Sigma) in dessicator and chromatographic purification, NMR, and mass spectrometric analysis), and GlcNAcβ3Man (DextraLabs, Reding, UK) by incubating the acceptor saccharide with β4Galactosyltransferase (bovine milk, Calbiochem., CA, USA) and UDP-Gal in presence of 20 mM MnCl₂ for several hours in 50 mM MOPS-NaOH pH 7.4. Hexasaccharide Galβ3GlcNAcβ3Galβ4GlcNAc⊕3Galβ4Glc (1 mg, from Dextra labs, UK)) was treated with 400 mU β3/6-galactosidase (Calbiochem., CA, USA) overnight as suggested by the producer to obtain GlcNAcβ3GlcNAcβ3Galβ4GlcNAcβ3Galβ4Glc, GlcAβ3Galβ4GlcNAcβ6GlcNAc, GlcAβ3Galβ4GlcNAcβ3Galβ4Glc were produced from Galβ4GlcNAcβ6GlcNAc, GalβB4GlcNAcβ3Galβ4Glc by transglycosylation catalysed by glucuronidase enzyme (bovine testes, Sigma) using GlcA-paranitrophenyl as donor substrate. Glc(A-methylamide)β3Galβ4GlcNAcβ6GlcNAc was obtained by methylamidation of the corresponding glucuronyloligosaccharide as described. The oligosaccharides were purified chromatographically and their purity was assessed by MALDI-TOF mass spectrometry and NMR. Galα3Galβ4GlcNAcβ3Galβ4Glc was from Dextra laboratories, Reading, UK. The glycolipid GlcAβ3Galβ4GlcNAcβ3Galβ4GlcβCer (Wako Pure Chemicals, Osaka, Japan) was reduced to Glcβ3Galβ4GlcNAcβ3Galβ4GlcβCer as described in Lanne et al 1995. The glycolipid derivative Glc(A-methylamide)_{β}3Galβ4GlcNAcβ3Galβ4GlcβCer was produced by amidatation of the carboxylic acid group of the glucuronic acid of GlcAβ3Galβ4GlcNAcβ3Galβ4GlcβCer as described in Lanne et al 1995.

Beta3-glucuronidation and amidation of Galβ84GlcNAcβ6GlcNAc

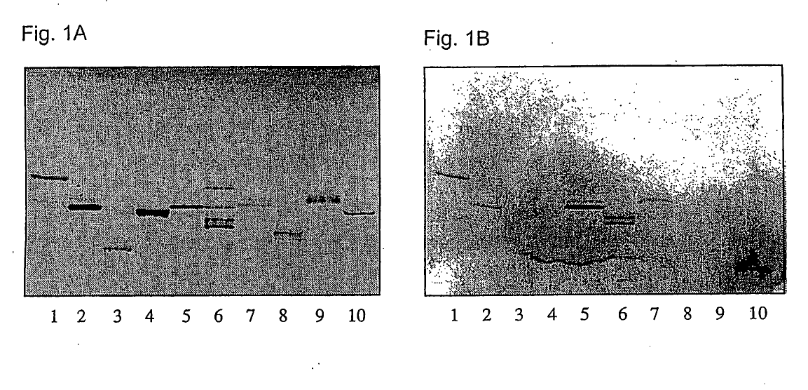

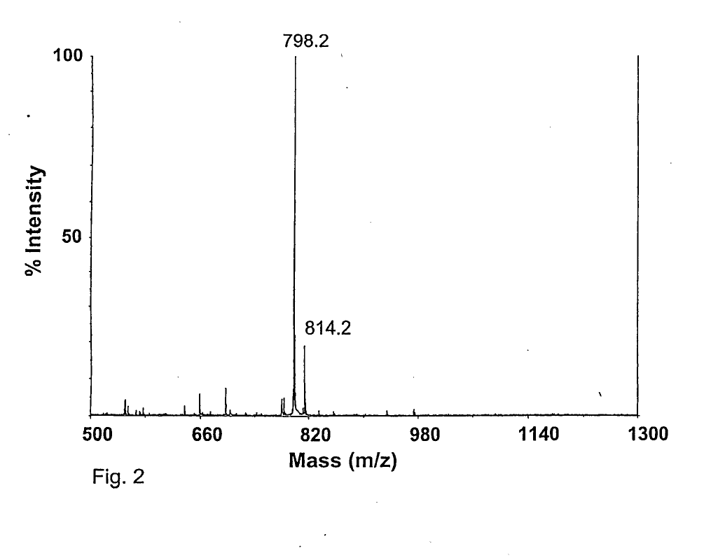

Galβ4GlcNAcβ6GlcNAc was β3-glucuronidated by transglycosylation using paranitrophenylglucuronide as the donor and bovine β1,3-glucuronidase as the enzyme. The product was purified by solid-phase extraction using a C-18 material, anion-exchange chromatography and gel filtration. The glucuronic acid unit in GlcAβ3Galβ4GlcNAcβ6GlcNAc was converted to glucuronmethylamide as follows: The tetrasaccharide was dissolved in pyridine containing 10% water, and 5-fold molar excess of HBTU (Benzotriazole-1-yl-1,1,3,3, tetramethyluroniumhexafluorophosphate) and DIPEA (N,N,-diisopropylethylamine) were added. 50-fold molar excess of methylamine was then added and the reaction was allowed to proceed for 48 h at room temperature. The methylamidated tetrasaccharide was puridied by gel filtration, anion-exchange chromatography as well as hydrophilic interaction chromatoraphy. MALDI-TOF mass spectrum of the purified product (FIG. 2) shows the expected signals: m/z 798.2 ([M+Na]⁺) and 814.2 ([M+K]⁺). The structure of the molecule was confirmed NMR-spectormetry. The glucuronic acid unit in GlcAβ3Galβ4GlcNAcβ6GlcNAc was also converted to glucuronamide. Essentially similar method was used as above, except that ammonia was used as the base. The structure of the molecule was confirmed by MALDI-TOF mass spectrometry and NMR-spectormetry-

### Example 2

Production of Amidated Chondroitin Type Oligosaccharides

Chondroitin sulphate A (Sigma) was converted to pyridinium salt by running the sample through cation exchange resin (hydrogen form), after which the solution was titrated to slightly basic pH with pyridine, and then dried in a vacuum sentrifuge. Desulphation was carried out by dissolving the dry sample to DMSO containing 10% methanol, and incubating this solution for 4 h at 80° C. DMSO was removed by extensive dialysis against water.

Oligosaccharides were produced from the desulphated material by acid hydrolysis in 0.5 M TFA at 60° C. for 18 h. In these conditions tetra- and hexasaccharides with GalNAc at their reducing end are effectively produced. Pure tetra- and hexasaccharides were isolated by use of gel filtration and anion-exchange chromatography. NMR-spectorometry reavealed oligosaccharide with non-reducing terminal GlcA and reducing terminal GalNAc. The hydrolysis method revealed to be surprisingly selective in cleaving GalNAcβ4GlcA-bond. The use of volatile acid allows use of oligosaccharide mixtures even without desalting, which is another benefit of using trifluoroacetic acid or similar strong carboxylic acids.

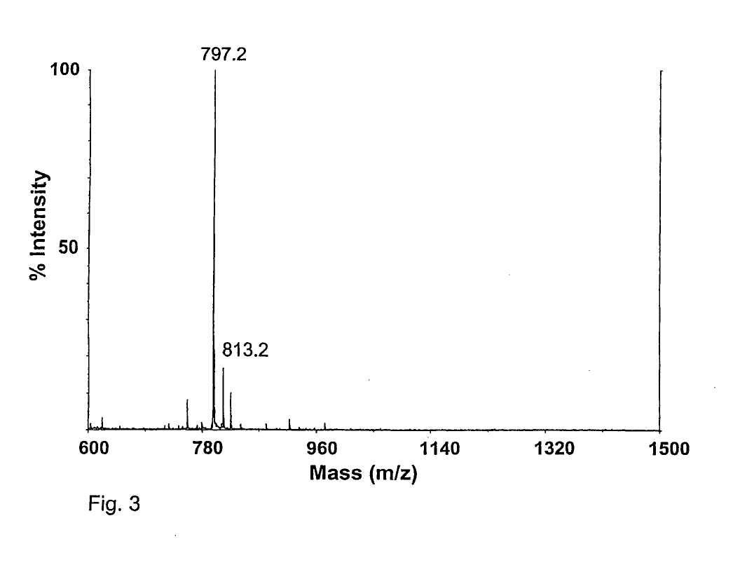

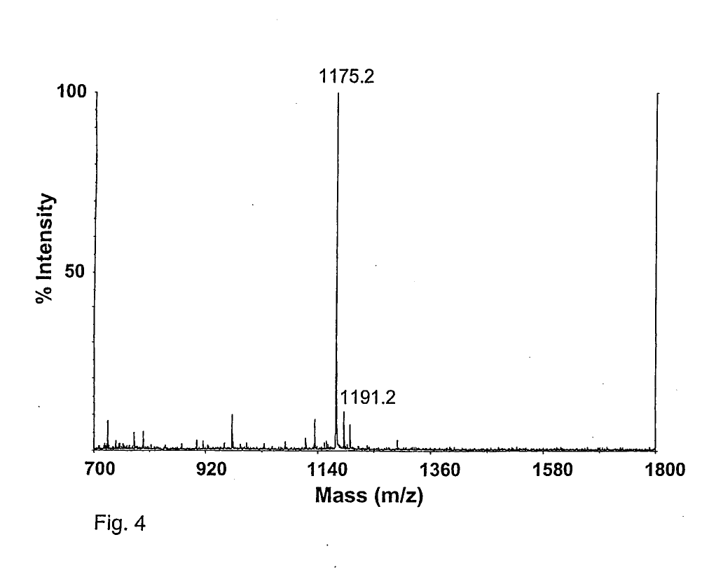

Amide groups were introduced to glucuronic acid residues of chondroitin tetrasaccharides and hexasaccharides by using HBTU activator and ammonia as described above. Pure amidated tetra- and hexasaccharides were isolated by use of gel filtration and anion-exchange chromatography. The MALDI-TOF mass spectra of the purified products (FIGS. 3 and 4) show the expected signals.

Alternatively, amide groups can be introduced to the desulphated chondroitin sulphate polymer, using the methodology essentially as described above. When using polymer in a preferred method amidation reagents are effectively removed by extensive dialysis, and modified oligosaccharides are produced by acid hydrolysis, as described above. The methods to degradate polysaccharide and amidate glucuronic acid residues described above are also used be (without desulphation step) for commercial hyaluronic acid (for example Sigma) or oligosaccharides such as GlcAβ3GlcNAcβ4GlcAβ3GlcNAc and GlcAβ3GlcNAcβ4GlcAβ3GlcNAcβ4GlcAβ3GlcNAc derived from hyaluronic acid (as above). Alternatively hyaluronidase (bovine, Sigma) can be used for production of chondroitin or chondroitin sulphate or hyaluronic acid oligosaccharides.

### Example 3

The binding of Helicobacter pylori (strain 032, several other strains were used in other experiments) to purified glycosphingolipids separated on thin-layer plates using the overlay assay is shown in FIG. 1 panels A and B. These results together with those from an additional number of purified glycosphingolipids are summarized in Table 1. The binding of Helicobacter pylori to neolactotetraosylceramide (lane 1) and the five- and six-sugar glycosphingolipids (lanes 5 and 6) derived from NeuGcα3Galβ4GlcNAcβ3Galβ4GlcNAcβ3Galβ4GlcβCer is identical to results above. Unexpectedly, however, binding was also found for GalNAcβ3Galβ4GlcNAcβ3Galβ4GlcβCer (x₂ glycosphingolipid, lane 7) and the de-fucosylated A6-2 glycosphingolipid GalNAcα3Galβ4GlcNAcβ3Galβ4GlcβCer (no. 12, Table 1). Together with the finding that Galα3Galβ4GlcNAcβ3Galβ4GlcβCer (B5 glycosphingolipid, lane 2) also is binding-active, these results suggest the possibility of cross-binding rather than the presence of multiple adhesins specific for each of these glycosphingolipids (see below). Furthermore, the only extension of the different five-sugar-containing glycosphingolipids just mentioned that was tolerated by the bacterial adhesin was Galβ4 to the thymus-derived GlcNAcβ3-terminated compound (lane 6). Other elongated structures, as the NeuAc-x₂ (lane 8) and GalNAcβ3-B5 (no. 25, Table 1), were thus all found to be non-binding. It may be further noticed that the acetamido group of the internal GlcNAcβ3 in B5 is, essential for binding since de-N-acylation of this moiety by treatment with anhydrous hydrazine leads to complete loss of binding (lane 3) as is the case also when neolactotetraosylceramide is similarly treated (no. 6, Table 1).

Cross-binding of five-sugar glycosphingolipids. In order to understand the binding characteristics of the different neolacto-based glycosphingolipid molecules used in this study the conformational preferences of active as well as inactive structures were investigated by molecular modeling. The four active structures all have neolacto cores which thus are terminated by GalNAcβ3, GalNAcα3, Galα3 and GlcNAcβ3, respectively. The minimum energy conformers of these structures were generated as described previously (Teneberg et al., 1996). Other minimum energy structures according to the invention are based on earlier results found in the literature (Bock et al., 1985; Meyer, 1990; Nyholm et al., 1989).

As mentioned above, the fact that there are four binding-active five-sugar glycosphingolipids (nos. 10-13, Table 1), all having a neolacto core, suggests that cross-binding to the same adhesin site may be the reason behind these observations. At first glance, however, it might seem surprising that the B5 glycosphingolipid, which differs at the terminal position in comparison with the five-sugar compound obtained from rabbit thymus, the former having a Galα3 and the latter a GlcNAcβ3, is equally active and should be included within the binding specificity of the neolacto series. Despite the fact that these two terminal saccharides differ also in their anomeric linkage it is seen that the minimum energy structures topographically are very similar, the differences being that Galα3 lacks an acetamido group, has the 4-OH in the axial position and its ring plane raised slightly above the corresponding plane in the five-sugar compound. However, neither the 4-OH position nor the absence/presence of an acetamido group appear to be crucial for binding to occur, since also the x₂ and defucosylated A6-2 glycosphingolipids, which are terminated by GalNAcβ3 and GalNAcα3, respectively, have similar affinities for the Helicobacter pylori adhesin. In the light of these findings also Galα3Galβ4GlcNAcβ3Galβ4GlcβCer, known from human erythrocytes, would be expected to bind the bacterial adhesin and also three other terminal monosaccharides in Helicobacter pylori binding epitopes are trisaccharide binding epitopes, namely GlcNAcα3Galβ4GlcNAc, Glcβ3Galβ4GlcNAc and Glcα3Galβ4GlcNAc. Such compounds are not known from human tissues so far, but could rather represent analogues of the natural receptor. Finally similar sequences with changes in the terminal monosaccharide position 6, (for example carboxylic acid, desulfo HNK-1, or amide derivative) and N-asetyl group on position 2 of Galβ4 were included to the family of Neolacto type receptors for H. pylori.

### Example 4

Analysis of neoglycolipids The oligosaccharides GlcNAcβ3Galβ4GlcNAc, GlcNAcβ3Galβ4GlcNAcβ6GlcNAc, Galα3Galβ4GlcNAcβ3Galβ4Glc, Glc(A-methylamide)β3Galβ4GlcNAcβ6GlcNAc, Galβ4GlcNAcβ6GlcNAc, GlcNAcβ3Galβ4GlcNAcβ6Gal GlcNAcβ3Galβ4GlcNAcβ6Galβ4Glc GlcNAcβ3Galβ4GlcNAcβ6GlcNAc, GlcNAcβ3Galβ4GlcNAcβ3Galβ4Glc, GlcAβ3Galβ4GlcNAc, 6GlcNAc, GlcAβ3Galβ4GlcNAcβ3Galβ4Glc, GlcNAcβ3Galβ4GlcNAcβ3Man, GalNAcβ4GlcNAcβ3Galβ4GlcNAc, Glc(A-NH₂)β3GalNAcβ4Glc(A-NH₂)β3GalNAc, GlcAβ3GalNAcβ4GlcAβ3GalNAcβ4GlcAβ3GalNAc and maltoheptaose (Sigma, Saint Louis, USA), wherein A-NH₂ indicates the glucuronamides CO—NH₂ and (A-methylamide) methylamide of the 6-position of glucuronic acid, —CO—NH—CH₃, were reductively aminated with 4-hexadecylaniline (abbreviation HDA, from Aldrich, Stockholm, Sweden) and/or with another lipid anchor NH₂—CH₂—CH₂—CH₂-amide-lysineamidated to two palmitates (custom product, Rapp polymere, Germany) by cyanoborohydride (Halina Miller-Podraza, to be published later). The products were characterized by mass spectrometry and were confirmed to be conjugates reductively aminated at the reducing end of the molecules to the lipid anchor/anchors. The neoglycolipids from oligosaccharides Galα3Galβ4GlcNAcβ3Galβ4Glc, and GlcNAcβ3Galβ4GlcNAcβ3Galβ4Glc, Galβ4GlcNAcβ6GlcNAc, GlcAβ3Galβ4GlcNAcβ6GlcNAc, GlcAβ3Galβ4GlcNAcβ3Galβ4Glc, GlcNAcβ3Galβ4GlcNAcβ3Man, GalNAcβ4GlcNAcβ3Galβ4GlcNAc, GlcAβ3GalNAcβ4GlcAβ3GalNAcβ4GlcAβ3GalNAc had clear binding activity and GlcNAcβ3Galβ4GlcNAcβ6GlcNAc, Glc(A-methylamide)β3Galβ4GlcNAcβ6GlcNAc, GlcNAcβ3Galβ4GlcNAcβ6Gal and Glc(A-NH₂)β3GalNAcβ4Glc(A-NH₂)β3 GalNAc derivatives had strong binding activity with regard to Helicobacter pylori in TLC overlay assay described above, while the GlcNAcβ3Galβ4GlcNAc, GlcNAcβ3Galβ4GlcNAcβ6Galβ4Glc GlcNAcβ3Galβ4GlcNAcα6GlcNAc and maltoheptaose neoglycolipids were weakly binding or inactive. The example shows that the terminal tri- or disaccharide epitopes are preferably represented β6-HexNAc(Gal, GlcNAc) linked as structures binding to Helicobacter pylori. The reducing end Glc-residue is probably not needed for the binding because the reduction destroys the pyranose ring structure of the Glc-residue. In contrast, the intact ring structure of reducing end GlcNAc is needed for good binding of the trisacharide GlcNAcβ3Galβ4GlcNAc.

### Example 5

### Analysis Novel Glycolipid Derivatives

The a biosynthetic precursor analog of NHK-1 glycolipid GlcAβ3Galβ4GlcNAcβ3Galβ4GlcβCer and novel glycolipids Glcβ3Galβ4GlcNAcβ3Galβ4GlcβCer and Glc(A-methylamide)β3Galβ4GlcNAcβ3Galβ4GlcβCer, Glc(A-ethylamide)β3 Galβ4GlcNAcβ3Galβ4GlcβCer, Glc(A-benzylamide)β3 Galβ4GlcNAcβ3Galβ4GlcβCer, and Glc(A-octadecylamide)β3Galβ4GlcNAcβ3Galβ4GlcβCer were tested in TLC overlay assay and were observed to be binding active with regard to Helicobacter pylori. Glc(A-methylamide) means glucuronic acid derivative wherein the carboxylic acid group is amidated with metylamine, in the other molecules glucuronic acid is analogously ethylamidated, benzylamidated and octadecylamidated to the carboxylic acid at 6-position. When comparing dilution series of glycolipids on TLC the Glcβ3Galβ4GlcNAcβ3Galβ4GlcβCer structure had strong binding towards H. pylori and Glc(A-methylamide)β3Galβ4GlcNAcβ3Galβ4GlcβCer had very strong binding to Helicobacter pylori. The methylamide and ethylamide derivatives were about equally strong binders and best among the four amide derivatives while binding to the benzylamide and octadecylamide glucuronylglycolips were also binding effectively.

Production of GlcAβ3Galβ4Glc(NAc) by Transglycosylation

The acceptor saccharide Galβ4Glc or Galβ4GlcNAc (about 10-20 mM) is incubated with 10 fold molar excess paranitrophenyl-beta-glucuronic acid and bovine liver β-glucuronidase (20 000U, Sigma) in buffer having pH of about 5 for two days at 37 degrees of Celsius stirring the solution. The product is purified by HPLC.

TABLE 1
Binding of Helicobacter pylori to glycosphingolipids separated on thin-layer chromatograms.

| | | | H. pylori | | |
|---|---|---|---|---|---|
| No. | Trivial name | Glycosphingolipid structure^{a} | binding^{b} | Source^{c} | References |
| 1 | Lactotri | GlcNAcβ3Galβ4Glcβ1Cer | − | RT | (Miller-Prodraza et al., 2001) |
| 2 | GgO3 | GalNAcβ4Galβ4Glcβ1Cer | (+) | GPE | (Yamakawa, 1966) |
| 3 | GgO3 (de-N-acylated) | GalNH₂β4Galβ4Glcβ1Cer | − | GPE^{e} | (Ångström et al., 1998) |
| 4 | Le^{y}-6 | Fucα2Galβ4(Fucα3)GlcNAcβ3Galβ4Glcβ1Cer | − | DSI | (McKibbin et al., 1982) |
| 5 | Neolactotetra | Galβ4GlcNAcβ3Galβ4Glcβ1Cer | (+) | HE^{f} | |
| 6 | Neolactotetra | Galβ4GlcNH₂β3Galβ4Glcβ1Cer | − | HB^{e} | |
| | (de-N-acylated) | | | | |
| 7 | GgO4 | Galβ3GalNAcβ4Galβ4Glcβ1Cer | + | HB^{g} | |
| 8 | GgO4 (de-N-acylated) | Galβ3GalNH₂β4Galβ4Glcβ1Cer | − | HB^{e} | (Ångström et al., 1998) |
| 9 | Le^{x}-5 | Galβ4(Fucα3)GlcNAcβ3Galβ4Glcβ1Cer | − | DSI | (Teneberg et al., 1996) |
| 10 | | GlcNAcβ3Galβ4GlcNAcβ3Galβ4Glcβ1Cer | + | RT^{d} | (Miller-Podraza et al., 2001) |
| 11 | x₂ | GalNAcβ3Galβ4GlcNAcβ3Galβ4Glcβ1Cer | + | HE | (Teneberg et al., 1996; Thorn et |
| | | | | | al., 1992) |
| 12 | | GalNAcα3Galβ4GlcNAcβ3Galβ4Glcβ1Cer | + | HE^{h} | |
| 13 | B5 | Galα3Galβ4GlcNAcβ3Galβ4Glcβ1Cer | + | RE | (Eto et al., 1968) |
| 14 | B5 (de-N-acylated) | Galα3Galβ4GlcNH₂β3Galβ4Glcβ1Cer | − | RE^{e} | |
| 15 | P₁ | Galα4Galβ4GlcNAcβ3Galβ4Glcβ1Cer | − | HE | (Naiki et al., 1975) |
| 16 | H5-1 | Fucα2Galβ3GlcNAcβ3Galβ4Glcβ1Cer | − | HM | (Karlsson and Larson, 1981a) |
| 17 | Le^{b}-6 | Fucα2Galβ3(Fucα4)GlcNAcβ3Galβ4Glcβ1Cer | − | HM | (Karlsson and Larson, 1981b) |
| 18 | H5-2 | Fucα2Galβ4GlcNAcβ3Galβ4Glcβ1Cer | − | HE | (Koscielak et al., 1973) |
| 19 | NeuAcα3-SPG | NeuAcα3Galβ4GlcNAcβ3Galβ4Glcβ1Cer | − | HE | (Ledeen and Yu, 1978) |
| 20 | NeuAcα6-SPG | NeuAcα6Galβ4GlcNAcβ3Galβ4Glcβ1Cer | − | HM | (Nilsson et al., 1981) |
| 21 | | Galβ4GlcNAcβ3Galβ4GlcNAcβ3Galβ4Glcβ1Cer | (+) | RT^{d} | (Miller-Podraza et al., 2001) |
| 22 | A6-2 | GalNAcα3(Fucα2)Galβ4GlcNAcβ3Galβ4Glcβ1Cer | − | HE | (Laine et al., 1974) |
| 23 | B6-2 | Galα3(Fucα2)Galβ4GlcNAcβ3Galβ4Glcβ1Cer | − | HE | (Koscielak et al., 1973) |
| 24 | NeuAc-x₂ | NeuAcα3GalNAcβ3Galβ4GlcNAcβ3Galβ4Glcβ1Cer | − | HE | (Watanabe and Hakomori, 1979) |
| 25 | | GalNAcβ3Galα3Galβ4GlcNAcβ3Galβ4Glcβ1Cer | − | RCC | (Thurin et al., 1989) |
| 26 | | Galβ4GlcNAcβ6(Galβ4GlcNAcβ3)Galβ4Glcβ1Cer | − | BB | (Teneberg et al., 1994) |
| 27 | | NeuGcα3Galβ4GlcNAcβ3Galβ4GlcNAcβ3Galβ4Glcβ1Cer | (+) | RT | (Lanne et al., 2001) |
| 28 | | Galα3Galβ4GlcNAcβ3Galβ4GlcNAcβ3Galβ4Glcβ1Cer | + | RT | (Lanne et al., 2001) |
| 29 | A7-2 | GalNAcα3(Fucα2)Galβ4(Fucα3)GlcNAcβ3Galβ4Glcβ1Cer | − | DSI | (Falk et al., 1979c) |
| 30 | B7-2 | Galα3(Fucα2)Galβ4(Fucα3)GlcNAcβ3Galβ4Glcβ1Cer | − | HE | |

Footnotes to Table 1
^{a}The glycosphingolipid shorthand nomenclature follows recent recommendations (Nomenclature of glycoproteins, 1988).
^{b}The following abbreviations are used for the glycosphingolipid sources: RT, rabbit thymus; HE, human erythrocytes; RE, rabbit erythrocytes; HM, human meconium; RCC, rat colon carcinoma; BB, bovine buttermilk; DSI, dog small intestine.
^{c}Definition of binding strength is as follows: + denotes a significant darkening of the autoradiogram with 4 μg applied on the TLC plate, (+) indicates a weak to intermediate darkening while a minus sign signifies no binding.
^{d}Prepared from No. 27 by mild acid hydrolysis and No. 10 by subsequent treatment with β-galactosidase.
^{e}Glycosphingolipid Nos. 3, 6, 8 and 14 were prepared from Nos. 2, 5, 7 and 13, respectively, by treatment with anhydrous hydrazine.
^{f}Prepared from no. 19 by neuraminidase treatment.
^{g}Prepared by mild acid hydrolysis of GM1 ganglioside from human brain.
^{h}Prepared from No. 22 by incubation in 0.05 M HCl at 80° C. for 2 h.

### REFERENCES

Andersson, B., Porras, O., Halson, L. Å., Lagergård, T., and Svanborg-Edén, C. (1986) J. Inf. Dis. 153, 232-7
Ångström, J., Teneberg, S., Abul Milh, M., Larsson, T., Leonardsson, I., Olsson, B.-M., Ölwegård Halvarsson, M., Danielsson, D., Näslund, I., Ljung, Å., Wadström, T. and Karlsson, K.-A. (1998) Glycobiology, 8, 297-309.
Ascencio, F, Fransson, L-Å. and Wadström, T. (1993) J. Med. Microbiol., 38, 240-244.
Appelmelk, B. J., Faller, G., Clayes, D., Kirchner, T., and Vandenbroucke-Grauls, C. M. J. E. (1998) Immol. Today 19, 296-299.
Avenaud, P., Marais, A., Monteiro, L., Le Bail, B., Biolac Saga, P., Balabaud, C., and Mégraud, F. (2000) Cancer 89, 1431-1439.
Axon, A. T. R. (1993) J. of Antimicrobial Chemotherapy, 32, 61-68.
Babior, B. M. (1978) N. Eng. J. Med., 298, 659-668.
Blaser, M. J. (1992) Eur. J. Gastroenterol. Hepatol., 4 (suppl 1), 17-19.
Bock. K., Breimer, M. E., Brignole, A., Hansson, G. C., Karlsson, K.-A., Larson, G., Leffler, H., Samuelsson, B. E., Strömberg, N., Svanborg-Edén, C. and Thurin, J. (1985) J. Biol. Chem., 260, 8545-8551.
Borén, T., Falk, P., Roth, K. A., Larson, G. and Normark, S. (1993) Science, 262, 1892-1895.
Castagliuolo, I., La Mont, J. T., Qiu, B., Nikulasson, S. T., and Pothoulakis, C. (1996) Gastroenterology 111, 433-438.
Castronovo, V., Colin, C., Parent, B., Foidart, J.-M., Lambotte, R., and Mahieu, P. (1989) J. Natl. Cancer Inst., 81, 212-216
Clausen, H., Levery, S. B., Kannagi, R. and Hakomori, S.-i. (1986) J. Biol. Chem., 261, 1380-1387.
Chmiela, M., Wadström, T., Folkesson, H., Planeta Malecka, I., Czkwianianc, E., Rechcinski, T., and Rudnicka, W. (1998) Immunol. Lett. 61, 119-125.
Claeys, D., Faller, G., Appelmelk, B. J., Negrini, R., and Kirchner, T. (1998) Gastroenterology 115, 340-347.
Correa, T. L., Fox, J., Fontham, E., Ruiz, b., Lin, Y., zaula, D., Taylor, N., Mackinley, D., deLima, E., Portilla, H., Zarama, G. (1990) Cancer 66, 596-574.
DeCross, A. J. and Marshall, B. J. (1993) Am. J. Med. Sci., 306, 381-392.
Dooley, C. P. (1993) Curr. Opin. Gastroenterol., 9, 112-117.
Dubois, M., Gilles, K. A., Hamilton, J. K., Rebers, P. A. and Smith, F. (1956) Analytical Chemistry 28, 350-356.
Dunn, B. E., Cohen, H. and Blaser, M. J. (1997) Clin. Microbiol. Rev., 10, 720-741.
Ernst, B., Hart, G. W., and Sinaÿ, P. (eds.) (2000) Carbohydrates in Chemistry and Biology, ISBN 3-527-29511-9, Wiley-VCH, Weinheim.
Eto, T., Ichikawa, Y., Nishimura, K., Ando, S. and Yamakawa, T. (1968) J. Biochem. (Tokyo), 64, 205-213.
Evans, D. G., Evans Jr, D. J., Molds, J. J., and Graham, D. Y. (1988) Infect. Immun., 56, 2896-06
Falk, K.-E., Karlsson, K-A. and Samuelsson, B. E. (1979a) Arch. Biochem. Biophys., 192, 164-176.
Falk K.-E., Karlsson, K.-A. and Samuelsson, B. E (1979b) Arch. Biochem. Biophys., 192, 177-190.
Falk, K.-E., Karlsson, K.-A. and Samuelsson, B. E. (1979c) Arch. Biochem. Biophys., 192, 191-202.
Farsak, B., Yildirir, A., Akyön, Y., Pinar, A., Öc, M., Böke, E., Kes, S., and Tokgözoglu, L. (2000) J. clin. Microbiol. 38, 4408-4411.
Folch, J., Lees, M., And Sloane-Stanley, G. H. (1957) J. Biol. Chem. 226, 497-509.
Fukuda, M. N., Dell, A., Oates, J. E., Wu, P., Klock, J. C. and Fukuda, M. (1985) J. Biol. Chem., 260, 1067-1082.
Handa, S. (1963) Jap. J. Exp. Med., 33, 347-360.
Hansson, G. C., Karlsson, K.-A., Larson, G., Strömberg, N. and Thurin, J. (1985) Anal. Biochem., 146, 158-163.
Hansson, G. C. and Karlsson, H. (1990) Methods Enzymol., 193, 733-738.
Hu, J., Stults, C. L., Holmes, E. H., and Macher, B. A. (1994) Glycobiology 4, 251-7.
Ilver, D., Arnqvist, A., Ogren, J., Frick, I. M., Kersulyte, D., Incecik, E. T., Berg, D. E., Covacci, A., Engstrand, L., and Boren T. (1998) Science, 279(5349), 373-377.
Ito, M. and Yamagata, T. (1989) Methods Enzymol., 179, 488-496.
Jassel, S. V., Ardill, J. E. S., Fillmore, D., Bamford, K. B., O'Connor, F. A., and Buchanan, K. D. Q. J. Med. 92, 373-377.
Karlsson, N. G., Olson, F. J., Jovall, P-Å, Andersch, Y., Enerbäck, L., and Hansson G. C. (2000) Biochem. J., 350, 805-814.
Karlsson, K.-A. (1987) Meth. Enzymol., 138, 212-220.
Karlsson, K.-A. (1989) Annu. Rev. Biochem., 58, 309-350.
Karlsson, K.-A. and Larsson, G. (1981a) J. Biol. Chem. 256, 3512-3524.
Karlsson, K.-A. and Larsson, G. (1981b) FEBS Lett., 128, 71-74.
Kerr, J. R., Al-Khattaf, A., Barson, A. J., and Burnie, J. P. (2000) Arch. Child. Dis., 83, 429-434
Koerner Jr, T. A. W., Prestegard, J. H., Demou, P. C. and Yu, R. K. (1983) Biochemistry, 22, 2676-2687.
Koscielak, J., Piasek, A., Gorniak, H., Gardas, A. and Gregor, A. (1973) Eur. J. Biochem., 37, 214-215.
Laine, R. A., Stellner, K. and Hakomori, S.-i. (1974) Meth. Membr. Biol., 2, 205-244.
Lanne, B., Uggla, L., Stenhagen, G., And Karlsson, K-A. (1995) Biochemistry 34, 1845-1850
Lanne, B., Miller-Podraza, H., Abul Milh, M., Teneberg, S., Uggla, L., Larsson, T., Leonardsson, I., Jovall, P.-Å., Bergström, J. and Karlsson, K.-A. (2001) manuscript in preparation
Larson, G., Karlsson, H., Hansson, G. C. and Pimlott, W. (1987) Carbohydr. Res., 161, 281-290
Ledeen, R. and Yu, R K; (1978) Res. Methods Neurochem., 4, 371-410
Lin, J.-T., Wang, J.-T., Wang, M.-S., Wu, M.-S. and Chen, C.-J. (1993) Hepato-Gastroenterol., 40, 596-599.
Lingwood, C. A., Huesca, M. and Kuksis, A. (1992) Infect. Immun., 60, 2470-2474.
Mayo, S. L., Olafsen, B. D. and Goddard III, W. A. (1990) J. Chem. Phys., 94, 8897-8909.
McKibbin, J. M., Spencer, W. A., Smith, E. L., Månsson, J. E., Karlsson, K-A, Samuelsson, B. E., Li, Y-T and Li, S. C. (1982) J. Biol. Chem., 257, 755-760.
Meyer, B. (1990) Topics Curr. Chem., 154, 141-208.
Miller-Podraza, H., Abul Milh, M., Bergström, J. and Karlsson, K.-A. (1996) Glycoconj. J., 13, 453-460.
Miller-Podraza, H., Bergström, J., Abul Milh, M. and Karlsson, K.-A. (1997a) Glycoconj. J., 14, 467-471.
Miller-Podraza, H., Abul Milh, M., Teneberg, S. and Karlsson, K.-A. (1997b) Infect. Immun., 65, 2480-2482.
Miller-Podraza, H., Abul Milh, M., Ångström, J., Jovall. P.-Å., Wilhelmsson, U., Lanne, B., Karlsson, H., and Karlsson, K.-A. (2001) manuscript in preparation.
Muzzarelli, R. A. A., Mattioli-Belmonte, M., Miliani, M., Muzzarelli, C., Gabbanelli, F., and Biagini, G. (2002) Carbohyrate Polym. 48, 15-21
Muzzarelli, R. A. A., Muzzarelli, C., Cosani, A., and Terbojevich, M. (1999) Carbohyrate Polym. 39, 361-367
Mysore, J. V., Wiggington, T., Simon, P. M., Zopf, D., Heman-Ackah, L. M. and Dubois, A. (1999) Gastroenterology, 117, 1316-1325
Naiki, M., Fong, J., Ledeen, R. and Marcus, D. M. (1975) Biochemistry, 14, 4831-4836.
Needs, P. W. and Selvendran, R. R. (1993) Carbohydr. Res., 245, 1-10.
Nilsson, H.-O., Taneera, J., Castedal, M., Glatz, E., Olsson, R., and Wadström, T. (2000) J. Clin. Microbiol. 38, 1072-1076.
Nilsson, O., Månsson, J.-E., Tibblin, E. and Svennerholm, L. (1981) FEBS Lett., 133, 197-200.
Nomenclature of glycoproteins (1988) J. Biol. Chem., 262, 13-18.
Nomura, A. and Stemmermann, G. N. (1993) J. Gastroenterol. Hepatol, 8, 294-303.
Nyholm, P. G., Samuelsson, B. E., Breimer, M. and Pascher, I. (1989) J. Mol. Recog., 2, 103-113.
Nyholm, P.-G. and Pascher, I. (1993) Biochemistry, 32, 1225-1234.
Ofek, I. and Sharon, N. (1988) Infect. Immun., 56, 539-547.
Pakodi, F., Abdel-Salam, O. M. E., Debraceni, A., and Mozsik, G. (2000) J. Physiol. (Paris), 94, 139-152.
Parsonnet, J., Friedman, G. D., Vandersteen, D. P., Chang, Y., Vogelman, J. H., Orentreich, N. and Sibley, R. K. (1991) N. Engl. J. Med., 325, 1127-31
Rautelin, H., Blomberg, B., Järnerot, G. and Danielsson, D. (1994a) Scand. J. Gastroenteol., 29, 128-132.
Rautelin, H., von Bonsdorff, C.-H., Blomberg, B. and Danielsson, D. (1994b) J. Clin. Pathol., 47, 667-669.
Rebora, R., Drago F., and Parodi, A. (1995) Dermatology 191, 6-8.
Saitoh, T., Natomi, H., Zhao, W., Okuzumi, K., Sugano, K., Iwamori, M. and Nagai, Y. (1991) FEBS Lett., 282, 385-387.
Samuelsson, B. E., Pimlott, W. and Karlsson, K.-A. (1990) Meth. Enzymol., 193, 623-646.
Sears, P., and Wong, C-H. (1996) Proc. Natl. Acad. Sci., 93, 12086-12093.
Simon, P. M., Goode, P. L., Mobasseri, A., and Zopf, D. (1997) Infect. Immun. 65, 750-757
Soltesz, V., Schalen, C. and Mårdh, P. A. (1988) Proceedings of the Fourth International Workshop on Campylobacter Infections (Kaijser, B. and Falsen, E., eds.) pp. 433-436, Goterna, Kungälv, Sweden.
Steininger, H., Faller, G., Dewald, E., Brabletz, T., Jung, A., and Kirchner, T. (1998) Virchows Arch. 433, 13-18.
Stellner, K., Saito, H. and Hakomori, S.-i. (1973) Arch. Biochem. Biophys., 155, 464-4.

Stroud, M. R., Handa, K., Salyan, M. E. K., Ito, K., Levery, S. B., Hakomori, S.-i., Reinhold, B. B. and Reinhold, V. N. (1996) Biochemistry, 35, 758-769.
Sung, J., Russell, R. I., Neyomans, Chan, F. K., Chen, S., Fock, K., Goh, K. L., Kullavanijaya, P., Kimura, K., Lau, C., Louw, J., Sollano, J., Triadiafalopulos, G., Xiao, S., Brooks, P. (2000) J. Gastroenterol. Hepatol., 15, Suppl: G58-68.
Teneberg, S., Ångström, J., Jovall, P.-Å. and Karlsson, K.-A. (1994) J. Biol. Chem., 269, 8554-8563.
Teneberg, S., Lönnroth, I., Torres Lopez, J. F., Galili, U., Ölwegård Halvarsson, M., Ångström, J. and Karlsson, K.-A. (1996) Glycobiology, 6, 599-609.
Thorn, J. J., Levery, S. B., Salyan, M. E. K., Stroud, M. R., Cedergren, B., Nilsson, B., Hakomori, S.-i. and Clausen, H. (1992) Biochemistry, 31, 6509-6517.
Thurin, J., Brodin, T., Bechtel, B., Jovall, P.-Å., Karlsson, H., Strömberg, N., Teneberg, S., Sjögren, H. O. and Karlsson, K.-A. (1989) Biochim. Biophys. Acta, 1002, 267-272.
Vivier, E., Sorrell, J. M., Ackerly M., Robertson M. J., Rasmussen R. A., Levine H., and Anderson P. (1993) J. Exp. Med., 178(6), 2023-33.
Waldi, D. (1962) in Dünnschicht-Chromatographie (Stahl, E., ed.) pp. 496-515, Springer-Verlag, Berlin.
Watanabe, K. and Hakomori, S.-i. (1979) Biochemistry, 18, 5502-5504.
Wotherspoon, A. C., Doglioni, C., Diss, T. C., Pan, L., Moschini, A., de Boni, M. and Isaacson; P. G. (1993) Lancet, 342, 575-577
Yamakawa, T. (1966) Colloq. Ges. Physiol. Chem., 16, 87-111.
Yang, H.-j. and Hakomori, S.-i. (1971) J. Biol. Chem., 246, 1192-1200.

1-67. (canceled)

68. A Helicobacter pylori binding substance comprising terminal oligosaccharide sequence
[Hex1(A)_{q1}(NAc)ᵣ₁α/β3]ₛGal(NAc)ᵣ₂β4Glc(A)_{q2}(NAc)ᵣ₃
wherein q1, q2, r1, r2, r3, and s are each independently 0 or 1 so that at least r2 or q2 is 1;
Hex1 is galactose (Gal), glucose (Glc) or mannose (Man);
and analogs or derivatives of said oligosaccharide sequence having binding activity to Helicobacter pylori for the prophylaxis or treatment of any condition due to the presence of Helicobacter pylori in a subject.

69. The Helicobacter pylori binding substance according to claim 68 further comprising β6Hex3(NAc)ᵣ₅ or β3Hex3(NAc)ᵣ₅ structure in the reducing end of the oligosaccharide sequence forming the following structure
[Hex1(A)_{q1}(NAc)ᵣ₁α/β3]ₛGal(NAc)ᵣ₂β4Glc(A)_{q2}(NAc)ᵣ₃β6/β3Hex3(A)ᵣ₄(NAc)ᵣ₅
wherein q1, q2, r1, r2, r3, s, and Hex1 are as defined in claim 68, r4 and r5 are independently 0 or 1; Hex3 is mannose (Man), galactose (Gal) or glucose (Glc).

70. A Helicobacter pylori binding substance comprising oligosaccharide sequence
Glc(A)_{q1}(NAc)ᵣ₁β3Galβ4Glc(NAc)ᵣ₃β6Hex3(NAc)ᵣ₅
wherein q1, r1, and r3 are as defined in claim 68, r5 and Hex3 are as defined in claim 69.

71. The Helicobacter pylori binding substance according to claim 68 wherein said oligosaccharide sequence is a natural type chondroitin sequence according to the following structure
[GlcAβ3]ₛGalNAcβ4GlcA[β3GalNAc]ᵤ
wherein s and u are as defied above with the proviso that either s or u is 1.

72. A Helicobacter pylori binding substance comprising at least one terminal oligosaccharide sequence selected from the group consisting of:
Glcβ3GalNAcβ4Glc,
Glcβ3GalNAcβ4Glcβ3GalNAcβ4Glc,
Glcβ3GalNAcβ4Glcβ3GalNAcβ4Glcβ3GalNAcβ4Glc,
Glcβ3GalNAcβ4Glcβ3GalNAc,
Glcβ3GalNAcβ4Glcβ3GalNAcβ4Glcβ3GalNAc,
Glcβ3GalNAcβ4Glcβ3GalNAcβ4Glcβ3GalNAcβ4Glcβ3GalNAc,
GlcAβ3GalNAcβ4GlcA,
GlcAβ3GalNAcβ4GlcAβ3GalNAcβ4GlcA,
GlcAβ3GalNAcβ4GlcAβ3 GalNAcβ4GlcAβ3GalNAcβ4GlcA,
Glcβ3Galβ3GalNAcβ4GlcAβ3GalNAcβ4GalNAcβ3GalNAcβ4GlcAβ3GalNAcβ4GlcA
GalNAcβ4GlcAβ3GalNAcβ4GlcA,
GalNAcβ4GlcAβ3GalNAcβ4GlcAβ3 GalNAcβ4GlcA,
GalNAcβ4GlcAβ3GalNAcβ4GlcAβ3GalNAcβ4GlcAβ3GalNAcβ4GlcA,
GalNAcβ4GlcAβ3GalNAc,
GalNAcβ4GlcAβ3GalNAcβ4GlcAβ3GalNAc,
GalNAcβ4GlcAβ3GalNAcβ4GlcAβ3GalNAcβ4GlcAβ3GalNAc
GalNAcβ4Glc, and
GalNAcβ4GlcA

73. Use of a Helicobacter pylori binding substance comprising terminal oligosaccharide sequence
[Hex1(A)_{q1}(NAC)ᵣ₁α/β3]ₛGal(NAc)ᵣ₂β4Glc(A)_{q2}(NAc)ᵣ₃
wherein q1, q2, r1, r2, r3, and s are each independently 0 or 1 so that at least r2 or q2 is 1;
Hex1 is galactose (Gal), glucose (Glc) or mannose (Man);
and analogs or derivatives of said oligosaccharide sequence having binding activity to Helicobacter pylori
for the production of a pharmaceutical composition for the treatment of any condition due to the infection of Helicobacter pylori.

74. A pharmaceutical composition comprising the substance according to claim 68 for the treatment of any condition due to the presence of Helicobacter pylori.

75. The pharmaceutical composition according to claim 74 for the treatment of chronic superficial gastritis, gastric ulcer, duodenal ulcer, gastric adenocarcinoma, non-Hodgkin lymphoma in human stomach, liver disease, pancreatic disease, skin disease, heart disease, or autoimmune diseases including autoimmune gastritis and pernicious anaemia and non-steroid anti-inflammatory drug (NSAID) related gastric disease, or for prevention of sudden infant death syndrome.

76. A nutritional additive or composition containing the substance according to claim 68.

77. The substance according to claim 68 for use in Helicobacter pylori binding assays.

78. A Helicobacter pylori binding substance comprising an oligosaccharide sequence according to Formula 9 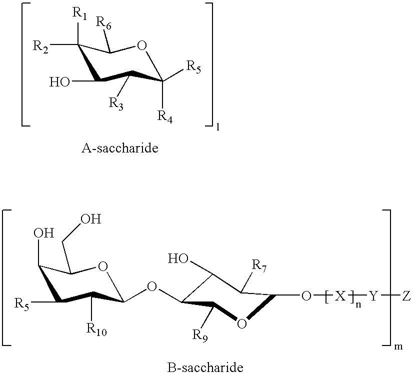
where integers 1, m, and n have values m=1, 1 and n are independently 0 or 1; R₁ is H and R₂ is OH, or R₁ is OH and R₂ is H, or R₁ is H and R₂ is a monosaccharidyl- or oligosaccharidyl-group, preferably a beta glycosidically linked galactosyl group, R₃ is independently —OH or acetamido (—NHCOCH₃) or an acetamido analogous group, R₇ is acetamido (—NHCOCH₃) or an acetamido analogous group; when 1=1, R₄ is —F and R₅ is oxygen linked to bond R₆ and forms a beta anomeric glycosidic linkage to saccharide B, or R₅ is —H and R₄ is oxygen linked to bond R₆ and forms an alpha anomeric glycosidic linkage to saccharide B; when 1=0, R₆ is —OH linked to B; X is monosaccharide or oligosaccharide residue, X is lactosyl-, galactosyl-, poly-N-acetyl-lactosaminyl, or part of an O-glycan or an N-glycan oligosaccharide sequence; Y is a spacer group or a terminal conjugate such as a ceramide lipid moiety or a linkage to Z; Z is an oligovalent or a polyvalent carrier; the oxygen linkage (—O—) between position C1 of the B saccharide and saccharide residue X or spacer group Y can be replaced by carbon (—C—), nitrogen (—N—) or sulphur (—S—) linkage; R₈ and R₉ are independently carboxylic acid amide, such as methylamide or ethyalamide, hydroxymethyl (—CH₂—OH) or a carboxylic acid group or an ester thereof, such as methyl or ethyl ester; R₃, R₇, and R₁₀ are independently hydroxyl, acetamido or acetamido group mimicking group, such as C₁₋₆ alkyl-amides, arylamido, secondary amine, preferentially N-ethyl or N-methyl, O-acetyl, or O-alkyl for example O-ethyl or O-methyl.

79. A functional food comprising substances according to claim 68.

80. The functional food according to claim 79, wherein said food is selected from the group consisting of animal feed, infant formula and beverage.

81. Helicobacter pylori binding substance
[Hex1(A)_{q1}(NAc)ᵣ₁y3]ₛ₁Gal(NAc)ᵣ₂β4Glc(A)_{q2}(NAc)ᵣ₃
wherein q1, q2r1, r2, r3, and s1, are each independently 0 or 1,
and Hex1, and Hex2 is a hexose structures, preferably galactose (Gal) or glucose (Glc), which may be further modified by the A and/or NAC groups; y is either alpha or beta indicating the anomeric structure of the terminal monosaccharide residue with the provisions that at least r2 is 1 or q2 is 1 and
that A indicates a glucuronamide when at least q1 or q2 is 1
or when s1 is 0, then
q2 is 1 and r2 is 0
or q2 and r2 and r3 are 1
or q2 and r2 are 1, r3 is 0 and A indicates a glucuronamide;
or when s is 1 then when r2 is 1 then at least q1 is 1 or q2 is 1
with the provision that the molecule does not comprise two non-derivatized β-linked glucuronic acid units.

82. A method for the treatment or prevention of a condition due to or caused by the presence of Helicobacter pylori, wherein a pharmaceutically effective amount of the substance according to claim 68 or 72 is administered to a subject in need of such treatment.

83. The method according to claim 82, wherein said condition is selected from the group consisting of chronic superficial gastritis, gastric ulcer, duodenal ulcer, gastric adenocarcinoma, non-Hodgkin lymphoma in human stomach, liver disease, pancreatic disease, skin disease, heart disease, or autoimmune diseases including autoimmune gastritis and pernicious anaemia and non-steroid anti-inflammatory drug (NSAID) related gastric disease, and sudden infant death syndrome.
